(19) United States
(12) Patent Application Publication
(10) Pub. No.: US 2025/0255235 A1
(43) Pub. Date: Aug. 14, 2025

(54) METHODS OF SELECTING ANIMALS OR PLANTS USING PHASED GENOTYPES

(71) Applicant: Inguran, LLC, Navasota, TX (US)

(72) Inventors: Yuri Tani Utsunomiya, São Paulo (BR); Adam Taiti Harth Utsunomiya, São Paulo (BR); Pablo Ross, Navasota, TX (US); Nader Deeb, Navasota, TX (US)

(73) Assignee: Inguran, LLC, Navasota, TX (US)

(21) Appl. No.: 19/054,237

(22) Filed: Feb. 14, 2025

Related U.S. Application Data
(63) Continuation of application No. 63/553,507, filed on Feb. 14, 2024.

(51) Int. Cl. A01H 1/04; A01H 1/02

(52) U.S. Cl. CPC A01H 1/045; A01H 1/02

(57) ABSTRACT

The invention encompasses methods of selecting an animal or plant, and producing progeny from the animal or plant, using segmented phased genotypes.

### REFERENCE TO RELATED APPLICATIONS

This application claims the benefit of U.S. Provisional Patent Application No. 63/553,507, filed Feb. 14, 2024, the entire disclosure of which is incorporated herein by reference.

### BACKGROUND OF THE INVENTION

Genomic mating can be viewed as an umbrella term in animal and plant breeding referring to the use of high density single-nucleotide polymorphism (SNP) marker data in the optimization of mating allocations and in the prediction of progeny genetics. Methods for the incorporation of SNP data into mating plans have been largely based on the maximization of genetic gain while restraining inbreeding and co-ancestry (Akdemir & Sánchez, 2016; Gorjanc & Hickey, 2018). The input data for these methods typically consist of a genomic relationship matrix (GRM) and estimated breeding values (EBVs) or predicted transmitting abilities (PTAs), with parent relationships in the GRM being used to predict the average inbreeding or co-ancestry and the average of parental EBVs or PTAs used to predict the mean of genetic merit in the progeny.

The use of genetic maps, phased genotypes, and haplotype data to estimate the effect of Mendelian sampling on progeny has been proposed to extend genomic mating beyond parent averages. Cole and VanRaden (2011) used SNP effects and phased genotypes to calculate upper and lower bounds of Mendelian sampling variance for economically important traits in dairy cattle and found that selection of chromosomes rather than animals may result in faster genetic progress. Cole and Null (2013) developed visualization tools for direct genomic values (DGVs) split into haplotypes and showed how these tools could impact selection programs by making quantitative genetics principles more tangible and intuitively connected to gametogenesis and molecular genetics. Segelke, et al. (2014) showed the power of simulating virtual gametes using phased genotypes and genetic maps to estimate the mean and variance of gametic breeding values. Within-family (co-)variability of marker effects has been explored by Bonk, et al. (2016), and Santos, et al. (2019; 2020) derived an analytical solution for the variance of gametic diversity as a function of allele substitution effects, linkage phase information at heterozygous loci and genetic maps. Although highly informative, the mentioned studies focused more on the variance of gametes and progenies, such that other distribution parameters such as higher-order moments (e.g., skewness and kurtosis) or even the distribution of gametes and progenies remained largely unexplored. Furthermore, in a scenario where multiple distribution statistics are to be simultaneously explored, it remains unclear if analytical methods (i.e., closed-form equations) will be more efficient and computationally tractable than sampling methods (i.e., Monte Carlo simulations).

Utsunomiya, et al. (2016) implemented an easy-to-use simulator that combines genetic maps and phased genotypes for the generation of virtual progenies, which allows for empirical distributions of any genetic parameter of interest (e.g., breeding values, inbreeding and ancestry proportions in crossbreeding) to be approximated. Despite its flexibility, applying the simulator to large-scale mating optimizations is not feasible if several million sire-dam pair combinations are to be tested. Ideally, sampling methods should be applied to individuals for the characterization of gamete distributions, with the distribution of progenies for specific sire-dam combinations being approximated from pre-computed individual-level gamete summary statistics. Although the simulator does implement self-fertilization in support of applications in plant breeding, which could be easily adapted to trick the program to perform sampling of gametes, applying it to large scale problems, such as those encountered in the dairy industry, is not feasible due to the computational burden of sampling thousands of progenies for thousands of sire-dam combinations.

### SUMMARY OF THE INVENTION

One embodiment of the invention comprises a method of producing animal or plant progeny comprising generating a phased genotype from a genotype of an animal or a plant in a population, wherein the phased genotype is comprised of a plurality of phased alleles; multiplying each phased allele in the plurality of phased alleles by a marker effect to produce a direct genomic value (DGV) for each phased allele; segmenting the phased genotype, wherein each of the segments is comprised of one or more phased alleles from the plurality of phased alleles; calculating a DGV for each of the segments to provide either a positive or negative DGV for each of the segments; selecting the animal or the plant as a parent based on the calculated DGV of one or more of the segments; and producing progeny from the animal or the plant. In a further embodiment, the number of segments and the size of each of the segments are determined heuristically. In a more specific embodiment, the number of segments and the size of each of the segments are a function of the effective number of independent chromosomal segments. In certain embodiments, the DGV for each of the segments is calculated using a sliding window method. In certain of these embodiments, the sliding window method comprises selecting a step size. The aforementioned method may in certain embodiments further comprise a step of displaying the segmented, phased genotype, wherein segments comprising a positive DGV are visually differentiated from segments comprising a negative DGV. In a more specific embodiment, the segments comprising a positive DGV are visually differentiated from the segments comprising a negative DGV using a color gradient produced from a set of colors, wherein each color in the set represents a percentile of the distribution of DGVs for a segment in the population.

Another embodiment of the invention comprises a method of producing animal or plant progeny comprising estimating skewness or kurtosis of a distribution of DGVs of an animal's gametes or a plant's gametes using i) phased genotypes or linkage phase information and ii) marker recombination rates; selecting the animal or the plant as a parent in a population based on the estimated skewness or kurtosis of the distribution; and producing progeny from the animal or the plant. In a further embodiment, the estimated skewness is positive. In another embodiment, the estimated kurtosis is less than 3. In yet another embodiment, the estimated kurtosis is greater than 3. In certain embodiments, the step of selecting the animal or the plant is based on the estimated kurtosis comprises calculating excess kurtosis. In further embodiments, the calculated excess kurtosis is negative or positive.

In yet another embodiment, the invention comprises a method of producing progeny from a male and a female of an animal or plant species comprising estimating skewness or kurtosis of a distribution of DGVs for the male's gametes and for the female's gametes using i) phased genotypes or linkage phase information and ii) marker recombination rates; estimating a distribution of progeny DGVs, EBVs or PTAs using the estimated skewness or kurtosis for the male's gametes and the estimated skewness or kurtosis for the female's gametes; selecting the male and the female as a mating pair based on the estimated distribution of progeny DGVs, EBVs or PTAs; and producing progeny from the mating pair. In a further embodiment, the step of selecting the male and the female as a mating pair based on the estimated distribution of progeny DGVs, EBVs or PTAs comprises calculating the probability of producing progeny having a DGV, EBV or PTA exceeding a threshold DGV, EBV, or PTA.

In another embodiment, the invention comprises a method of producing progeny from a first individual and a second individual of an animal or plant species comprising estimating skewness or kurtosis of a distribution of DGVs for the first individual's gametes and for the second individual's gametes using i) phased genotypes or linkage phase information and ii) marker recombination rates; estimating a distribution of progeny DGVs, EBVs, or PTAs using the estimated skewness or kurtosis for the first individual's gametes and the estimated skewness or kurtosis for the second individual's gametes; selecting the first individual and the second individual as a mating pair based on the estimated distribution of progeny DGVs, EBVs, or PTAs; and producing progeny from the mating pair. In a further embodiment, the step of selecting the first individual and the second individual as a mating pair based on the estimated distribution of progeny DGVs, EBVs, or PTAs comprises calculating the probability of producing progeny having a DGV, EBV, or PTA exceeding a threshold DGV, EBV, or PTA.

An additional embodiment of the invention comprises a method of producing progeny from a male and a female of an animal or plant species comprising generating a phased genotype from a genotype of the male and a phased genotype from a genotype of the female, wherein each of the phased genotypes is comprised of a plurality of phased alleles; for each of the phased genotypes, multiplying each phased allele in the plurality of phased alleles by a marker effect to produce a direct genomic value (DGV) for each phased allele; segmenting each of the phased genotypes, wherein each segment is comprised of one or more phased alleles from the plurality of phased alleles; for each of the phased genotypes, calculating a DGV for each of the segments to provide either a positive or a negative DGV for each of the segments; selecting the male and the female as a mating pair based on the calculated DGV of one or more segments from the male and one or more segments from the female; and producing progeny from the mating pair. In a further embodiment of the method, in the step of selecting the male and the female as a mating pair, the one or more segments from the male and the one or more segments from the female are located on the same corresponding chromosome pair in the male and the female and i) the calculated DGV of the one or more segments from the male is a positive DGV, and the calculated DGV of the one or more segments from the female is a negative DGV or ii) the calculated DGV of the one or more segments from the male is a negative DGV, and the calculated DGV of the one or more segments from the female is a positive DGV.

Another embodiment of the invention comprises a method of producing progeny from a first individual and a second individual of an animal or plant species comprising generating a phased genotype from a genotype of the first individual and a phased genotype from a genotype of the second individual, wherein each of the phased genotypes is comprised of a plurality of phased alleles; for each of the phased genotypes, multiplying each phased allele in the plurality of phased alleles by a marker effect to produce a direct genomic value (DGV) for each phased allele; segmenting each of the phased genotypes, wherein each segment is comprised of one or more phased alleles from the plurality of phased alleles; for each of the phased genotypes, calculating a DGV for each of the segments to provide either a positive or a negative DGV for each of the segments; selecting the first individual and the second individual as a mating pair based on the calculated DGV of one or more segments from the first individual and one or more segments from the second individual; and producing progeny from the mating pair. In a further embodiment of the method, in the step of selecting the first individual and the second individual as a mating pair, the one or more segments from the first individual and the one or more segments from the second individual are located on the same corresponding chromosome pair in the first individual and the second individual and i) the calculated DGV of the one or more segments from the first individual is a positive DGV, and the calculated DGV of the one or more segments from the second individual is a negative DGV or ii) the calculated DGV of the one or more segments from the first individual is a negative DGV, and the calculated DGV of the one or more segments from the second individual is a positive DGV.

Given the ubiquity of Mendelian inheritance in the natural world, it will be understood by those of ordinary skill in the art that the various embodiments of the invention can be applied across animal and plant species. In certain embodiments, the invention may be used with non-human mammalian species, including but not limited to bovine, canine, caprine, cervine, cetacean, equine, feline, galline, murine, ovine, piscine, and porcine species. In a particular embodiment, the invention may be used with Bos taurus, Bos indicus, and Sus scrofa animals. In another embodiment, the invention may be used with dairy cattle and, in a particular embodiment, Holstein dairy cattle.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows allele substitution effect solutions for milk, fat, and protein yield in Holstein cattle.

FIGS. 2A to 2C show regional plots of major QTLs on autosomes 5, 6 and 14, respectively, affecting milk, fat, and protein yield in Holstein cattle.

FIG. 3A shows chromosomal painting of two animals with extreme merits for fat yield.

FIG. 3B shows the gamete variation by chromosome of two animals with extreme merits for fat yield.

FIG. 3C shows the gamete variation by whole genome of two animals with extreme merits for fat yield.

FIG. 4 shows the distribution of gametic skewness and (excess) kurtosis for milk, fat, and protein.

FIG. 5 shows the means (bars) and standard deviations (whiskers) for the percentage of total gametic variance explained by recombinant haplotypes at specific chromosome pairs.

FIG. 6 shows means (bars) and standard deviations (whiskers) for the relative change in gametic skewness caused by recombinant haplotypes at specific chromosome pairs.

FIG. 7 shows means (bars) and standard deviations (whiskers) for the relative change in gametic excess kurtosis caused by recombinant haplotypes at specific chromosome pairs.

FIG. 8A shows chromosomal paintings for two animals with opposing skewness values for milk yield.

FIG. 8B shows the gamete variation by chromosome of two animals with opposing skewness values for milk yield.

FIG. 8C shows the gamete variation by whole genome of two animals with opposing skewness values for milk yield.

FIG. 9 shows realized vs predicted progeny distributions for milk, fat and protein yield in 122 families.

FIG. 10 shows gamete distributions and predicted progeny distributions for milk, fat and protein yield in a family including 42 offspring.

FIG. 11 shows the distribution of gametic skewness for different simulated distributions of SNP effects.

FIG. 12 shows the distribution of gametic excess kurtosis for different simulated distributions of SNP effects.

FIG. 13 is an example of a graphical display of segmented phased genotypes from two different animals.

FIG. 14 shows one embodiment of the invention using genomic complementarity.

### DETAILED DESCRIPTION OF THE INVENTION

One aspect of the invention encompasses a framework for displaying genomic values decomposed into haplotype-specific effects, hereafter referred to as “chromosomal painting.” In certain embodiments, chromosomal painting can be used as the basis for the prediction of distributions of gamete-specific genomic values. A particular embodiment of the invention provides derivations for the four first moments (mean, variance, skewness, and kurtosis) of the distribution of gamete direct genomic values (DGVs) as a function of recombination rates, linkage phase and marker (e.g., SNP) effects, and moments of the distribution of progeny DGVs as a function of the moments of parental gamete DGVs. In a further aspect of the invention, these moments can be used to estimate the distributions of progeny DGVs without the need for a second sampling step.

For purposes of the invention, the term “direct genomic value” (DGV) means the sum of marker effects for an animal (or for a plant, a chromosome, a chromosomal segment or a marker) for a particular trait. The term “estimated breeding value” (EBV) for purposes of the invention encompasses a genomic estimated breeding value (GEBV). The sum of an animal's (or a plant's, a chromosome's, a chromosomal segment's, or a marker's) DGV and polygenic effects (i.e., genetic effects not captured by markers) for a particular trait yields the animal's (or the plant's, the chromosome's, the chromosomal segment's, or the marker's) EBV for the trait. The animal's (or the plant's, the chromosome's, the chromosomal segment's, or the marker's) “predicted transmitting ability” (PTA) for a particular trait is one-half of its EBV for the trait. The term “expected progeny difference” (EPD) is equivalent to a PTA for purposes of the invention.

In the context of the invention, “moments” of a distribution refers to numerical values that provide information about the shape and characteristics of the distribution. Generally, the first moment (k=1) is the mean of the distribution, typically denoted as μ; the second moment (k=2) is the variance of the distribution, typically denoted as σ² (i.e., standard deviation squared); the third moment (k=3) is the skewness of the distribution; and the fourth moment (k=4) is the kurtosis (or peakedness) of the distribution.

The term “marker” means a detectable variation in a DNA sequence with a known location on a chromosome and can be comprised of a single nucleotide or multiple nucleotides. Markers include, but are not limited to, single nucleotide polymorphisms (SNPs), which are single base variants, microsatellite markers, insertion-deletion variants, inversion variants and copy number variants.

The term “allele” refers to one of the possible variants for a particular genetic marker.

The term “marker effect” refers to the correlation between the presence of a particular allele and the phenotypic expression of a particular trait. A marker effect can be determined by substituting one allele for another and then determining if there is a change in the phenotypic mean.

The term “direct genomic value” or “DGV” refers to the estimated effect of an individual's genome on a quantitative trait based on the similarity between the individual and a reference population with known phenotypes and genotypes.

The term “genotyping” refers to the process of determining the identity of a nucleotide at at least one position within the genome of an individual.

The term “genotype” refers to a determined identity of a nucleotide at at least one position within the genome of an individual.

The term “phased genotype” refers to a genotype of a haplotype of an individual.

The term “haplotype” refers to one or more alleles in an individual that are inherited from a particular parent of the individual.

For purposes of the invention, the term “linkage phase” refers the physical arrangement of linked genes (or markers) in a chromosome. When linked reference alleles for two genes or markers are located on the same homologous chromosome and the corresponding linked non-reference alleles for the two genes or markers are located on the other corresponding chromosome, the two genes or markers are said to be “in-phase.” Conversely, when a reference allele for a gene or marker is located on the same homologous chromosome as a linked non-reference allele of a second gene or marker, the two genes or markers are said to be “out of phase.” The term “linkage phase information” encompasses an indicator variable that assumes a positive value if alleles for two genes or markers are in-phase (for example, +1) or, conversely, a negative value if alleles for two genes or markers are out of phase (for example, −1).

Broadly, the invention allows a user to estimate and visualize the distribution of genomic values in three levels relevant to breeding programs: phased genotypes (i.e., one or both of an individual's haplotypes for a chromosome pair), gametes, and progenies. In particular, the invention provides a method for estimating and visualizing genomic values of an animal's chromosomes and chromosomal segments (which encompasses segments comprising a single marker).

### Genotyping

In one aspect of the invention, extracted and/or amplified DNA from cells from an animal may be genotyped using genomic single nucleotide polymorphism (SNP) arrays or chips, which are readily available for various species of animals from companies such as Illumina and Affymetrix. Alternatively, an entire genome, or a portion of a genome, can be sequenced using methods well-known in the art, such as those described below. Low density and high density chips are contemplated for use with the invention, including SNP arrays comprising from 3,000 to 800,000 SNPs. By way of example, a “50K” SNP chip can determine the identity of approximately 50,000 SNPs in an individual and is commonly used in the livestock industry to genotype individuals and to determine genetic merit or genomic estimated breeding values.

In certain embodiments of the invention, nucleic acid is extracted from cells and then sequenced using any method known in the art, including, but not limited to, Sanger sequencing and high throughput sequencing, which includes next generation (short read) sequencing and third generation (long read) sequencing. In one embodiment of the invention, one read with short read sequencing comprises approximately 100 to 300 base pairs, and one read with long read sequencing comprises approximately 10,000 or more base pairs. Nonlimiting examples of sequencing methods for use in the invention include single-molecule real time sequencing, ion semiconductor sequencing, pyrosequencing, sequencing by synthesis, combinatorial probe anchor synthesis, sequencing by ligation, nanopore sequencing, massively parallel signature sequencing, polony sequencing, DNA nanoball sequencing, heliscope single molecule sequencing, and sequencing using droplet based microfluidics or digital microfluidics.

By way of example only, the following DNA extraction and amplification procedure may be used in certain embodiments of the invention. One skilled in the art will know that variations on this method exist and that this method should not be construed to limit the functionality or scope of the current invention. This method is illustrative only.

1.5 ml tubes containing a cell suspension of cells from an individual are spun at ≥10000×g in a microcentrifuge for 45 seconds to pellet the cells. The suspension solution is pipetted off carefully so as to not remove the pelleted cells. Approximately 50 μl of suspension solution is left in each tube. The tubes are then vortexed for 10 seconds to resuspend the cell pellets. 300 μl of Tissue and Cell Lysis Solution (Epicentre; Madison Wisconsin; Catalog #MTC096H) containing 1 μl of Proteinase K (Epicentre; Madison Wisconsin; at 50 μg/μl; Catalog #MPRK092) is then added to each tube and mixed. The tubes are incubated at 65° C. for 30 minutes and vortexed at 15 minutes. The samples are cooled to 37° C. Afterwards 1 μl of 5 mg/μl RNase A (Epicentre; Madison Wisconsin; at 5 mg/ml; Catalog #MPRK092) is added to each sample and then mixed. The samples are then incubated at 37° C. for 30 minutes. The samples are then placed in a 4° C. cooler for 5 minutes. 175 μl of MPC Protein Precipitation Reagent (Epicentre; Madison Wisconsin; Catalog #MMP095H) is added to each sample, and the samples vortexed vigorously for 10-15 seconds. The samples are centrifuged in order to pellet debris for 8 minutes at ≥10000×g. The supernatant is transferred to a clean microcentrifuge tube. 600 μl of cold (−20° C.) isopropanol is added to the supernatant. Each tube is then inverted 30-40 times. The DNA is pelleted by centrifugation for 8 minutes in a microcentrifuge at ≥10000×g. The isopropanol is poured off without dislodging the DNA pellet. The pellet is rinsed once with 70% ethanol and then the ethanol is carefully poured off so as not to disturb the DNA pellet. The residual ethanol is removed with a pipet, and the DNA pellet is allowed to air dry in the microcentrifuge tube. Once dried, the DNA pellet is resuspended in 20 μl Tris-EDTA.

In certain embodiments of the invention, DNA from cells can be extracted using the Purelink Genomic Kit Cat #K1820-00 (Invitrogen). In further embodiments, once the DNA is extracted, it can be put through a whole genome amplification protocol using the Illustra Genomiphi V2 DNA amplification kit (GE Lifesciences), which uses the phi29 DNA polymerase to amplify the genome.

### Generating a Phased Genotype

One aspect of the invention encompasses generating a phased genotype (i.e., one or more haplotypes) from a genotype of an animal or a plant in a population, wherein the phased genotype is comprised of a plurality of phased alleles, and a further embodiment comprises multiplying each phased allele in the plurality of phased alleles by a marker effect to produce a direct genomic value (DGV) for each phased allele. Once this is done, one can calculate a DGV for a segment of the phased genotype. When done for a plurality of segments covering the length of a haplotype of a chromosome, for example, one can proceed with visually differentiating the segments by assigning a color to each segment based on each segment's contribution (e.g., a positive or negative contribution) to the animal's or plant's DGV. Genotypes may be phased using the population-based method implemented in Eagle v2.4.1 (Loh et al., 2016).

In a particular embodiment of the invention (i.e., chromosomal painting), chromosomal segments or whole chromosomes comprising a positive DGV are visually differentiated from segments or chromosomes comprising a negative DGV in a graphical display of a phased genotype. In one embodiment of the invention, segments comprising a positive DGV are visually differentiated from segments comprising a negative DGV using a color gradient produced from a set of colors, wherein each color in the set represents a percentile of the distribution of DGVs for a segment in the population. Any suitable color system known in the art, including but not limited to RGB systems such as the hue, saturation and value system (“HSV”) and the hue, saturation and lightness system (“HSL”) may be used in the invention, where HSV or HSL coordinates define a particular color. For example, in one embodiment of the invention using HSV, all segments representing positive DGVs can be assigned a particular hue and segments representing different positive DGVs can have a different saturation or value, while all segments representing negative DGVs can be assigned a different particular hue and segments representing different negative DGVs can have a different saturation or value. In a particular embodiment, for example, all segments representing positive DGVs can be assigned a blue hue, while segments representing increasingly positive DGVs (for example, DGVs going from 1 to 2) can correspondingly be assigned the blue hue with an increasing saturation or value. Similarly, in this example, all segments representing negative DGVs can be assigned a red hue, while segments representing increasingly negative DGVs (for example, DGVs going from −1 to −2) can correspondingly be assigned the red hue with an increasing saturation or value. In an additional embodiment of the invention, it is contemplated that DGVs of chromosomal segments or whole chromosomes are visually differentiated from one another by altering one or more of the hue, saturation and value in the HSV system or one or more of the hue, saturation and lightness in the HSL system.

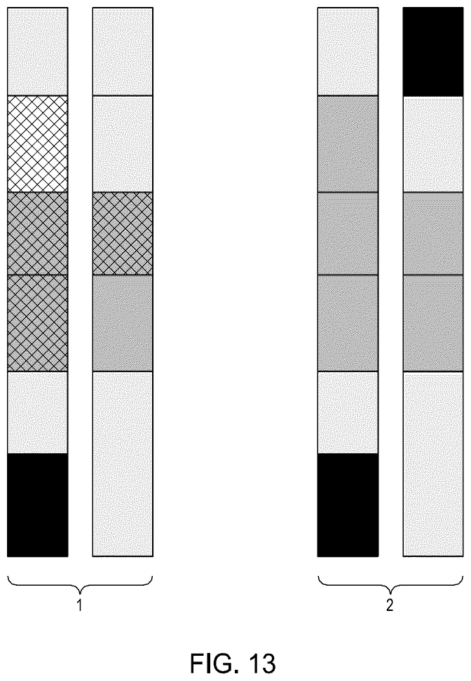

Alternatively, segments comprising a positive DGV may be visually differentiated from segments comprising a negative DGV using any technique for visually differentiation in a graphical display, such as by using cross-hatching or another type of pattern to denote segments with negative DGVs. Furthermore, greyscale can be used to denote the relative positivity or negativity of a segment. For example, referring to FIG. 13, in the phased genotype comprising negative segment DGVs (1), cross-hatching is used to differentiate segments comprising negative DGVs (i.e., segments comprising cross-hatching) from segments comprising positive DGVs (i.e., solid segments/segments without cross-hatching). Additionally, with respect to the embodiment depicted in FIG. 13, greyscale is used to indicate relative DGV values, with darker segments without cross-hatching indicating more positive DGVs and darker segments with cross-hatching indicating more negative DGVs. By visually differentiating chromosomal segments (or chromosomes) comprising positive and negative DGVs in a graphical display of a phased genotype, a breeder is able to quickly determine if an animal or a plant's genome is complementary to another's by visual inspection of the graphical display, i.e., a DGV of a chromosomal segment or chromosome from the first animal or plant is positive, while the corresponding chromosomal segment or chromosome from the other animal or plant is negative. Accordingly, an animal that would otherwise not be selected as a parent in a mating pair using conventional methods of selection based on estimated breeding values may be selected as a parent using the invention based on complementarity of chromosomal segments or chromosomes, e.g., the DGVs of certain chromosomal segments are positive in a relatively low genetic merit animal, while the DGVs of the corresponding segments in a high genetic merit prospective mate are negative. Referring to FIG. 13, the phased genotype comprising negative segment DGVs (1) may be from a high genetic merit animal, while the phased genotype comprising only positive segment DGVs (2) may be from a low genetic merit animal. In such a situation, a breeder may mate the two animals due to the complementarity of the segments.

Relative to animal i and marker j, \hat{α}ⱼ is the estimated average effect of replacing allele A by an alternative allele B (i.e., allele substitution effect), and mᵢⱼ is the genotype coded as the number of copies of allele B centered on the genotypic mean, i.e., (0−2pⱼ), (1−2pⱼ) and (2−2pⱼ) for allele combinations AA, AB and BB, respectively, where pⱼ is the frequency of allele B. The DGV of an animal can be expressed as:

$${DGV}_{i} = {\sum }_{j = 1}^{N} {m}_{ij} {\hat{α}}_{j}$$

Where N is the total number of markers. It follows that the contribution of a parent haplotype transmitting allele A is half of that of genotype AA, that is (0−pⱼ)αⱼ. Conversely, a parent haplotype transmitting allele B will contribute with (1−pⱼ)αⱼ. Therefore, for a binary coding representing the presence (1) or absence (0) of allele B, the contributions along a haplotype can be easily mapped by centering the indicator variable of each marker on the B allele frequency and multiplying it by the substitution effect. Furthermore, the contribution of specific haplotype segments to the DGV can be readily calculated by summing up (0−pⱼ)αⱼ and (1−pⱼ)αⱼ values over the inquired segment.

One way to determine a suitable segment size for use in chromosomal painting is N/Mₑ, where Mₑ is the estimated number of independent chromosomal segments. Following Goddard (2009), Mₑ can be approximated as 2NₑL/ln(4NₑL), where Nₑ is the effective population size and L is the autosomal genome size in Morgans. Alternatively, a suitable segment size can be determined heuristically. In one embodiment of the invention, a sliding window method can be utilized in order to calculate the DGV for each segment once a segment size has been determined.

### Distribution of Gametes

Considering a single chromosome pair, in one embodiment of the invention, the pseudo-code for a virtual gamete simulation algorithm is:

```
Sample the recombination number z from Z~Poisson(L_{chr})
Sample one of the parental haplotypes from h ∈ {1,2}
If z > 0:
  Sample z indices from i ∈ {1,2 ... N_{chr}} with
  probabilities ρ₁, ρ₂ ... ρ_{N}_{_{chr}}
  Append the last index (N_{chr}) to the list
  Initialize j = 1
  For each index k:
    Copy the segment [j; k] from haplotype h
    Set h as the other parental haplotype
    Set j = k + 1
Else:
  Copy the segment [1; N] from haplotype h
```

Where L_{chr} is the chromosome length in Morgans and ρ is the recombination rate. The algorithm is then looped by chromosome to extend simulations to the whole genome. The DGV of each simulated gamete is obtained by centering the resulting haplotype vector on allele frequencies and multiplying it by the vector of allele substitution effects.

For each chromosome separately and for the whole genome, one can compute and store percentiles and the four first moments of the distribution of DGVs in gametes. These moments can be estimated as:

$$\frac{1}{n} {\sum }_{i = 1}^{n} {( \frac{D G {V}_{i} - c}{q} )}^{k}$$

Where DGVᵢ is the genomic value of virtual gamete i, c is a constant, q is a scaling parameter and k is a power parameter. For the four moments we have: mean {c=0, q=1, k=1}, variance {c=x̅, q=s, k=2}, skewness {c=x̅, q=s, k=3} and kurtosis {c=x̅, q=s, k=4}, where x is the sample mean and s is the sample standard deviation. The mean informs central tendency, the variance quantifies dispersion around the mean, the skewness measures symmetry around the mean, and the kurtosis represents tail-heaviness or the tendency to produce outliers, which in some cases may also reflect the “peakedness” or “flatness” of a distribution, or the amount of density or mass found within the “shoulders” of the distribution (i.e., area within one standard unit of the mean). Using convergence plots, it was determined that in at least one embodiment of the invention, a good compromise between sampling error and computing time is 3,000 simulated gametes per animal.

### Distribution of Progenies

An offspring or progeny is formed by uniting paternal and maternal gametes, such that its genomic value is simply the sum of the inherited paternal and maternal haplotypes. Therefore, the moments of the progeny DGV distribution may be obtained as a function of the moments of the distributions of parental gametes as follows:

$${μ}_{p r o g e n y} = {μ}_{s i r e} + {μ}_{d a m}$$

$${σ}_{p r o g e n y}^{2} = {σ}_{s i r e}^{2} + {σ}_{d a m}^{2}$$

$${S}_{p r o g e n y}^{3} = \frac{{S}_{s i r e}^{3} {σ}_{s i r e}^{3} + {S}_{d a m}^{3} {σ}_{d a m}^{3}}{{σ}_{p r o g e n y}^{3}}$$

$${K}_{p r o g e n y}^{4} = \frac{{K}_{s i r e}^{4} {σ}_{s i r e}^{4} + {K}_{d a m}^{4} {σ}_{d a m}^{4} + 6 {σ}_{s i r e}^{2} {σ}_{d a m}^{2}}{{σ}_{p r o g e n y}^{4}}$$

Where S³ is the skewness and K⁴ is the kurtosis. Although skewness and kurtosis are not typically represented with superscripts 3 and 4, one may use this notation in this embodiment to make a distinction of these parameters with coskewness and cokurtosis discussed above. To convert progeny DGV to genomic PTA, the formulae may be adapted by replacing the mean by the parent average and dividing the variance by 4. Once the moments are obtained, the cumulants (κ) of the distribution may be computed as:

κ₁=μ

κ₂=σ²

κ₃=S³σ³

κ₄=K⁴*σ⁴

Where K⁴*=K⁴−3 is the excess kurtosis. Assuming unimodality and provided the cumulants above, probability and cumulative density functions may be approximated via the Edgeworth expansion, and quantiles and random draws from the distribution may be approximated using the Cornish-Fisher expansion (Pav, 2017).

### Allele Substitution Effects

Allele substitution effects for a trait may be obtained by regressing PTAs onto marker genotypes using:

$$y = 1 μ + g + e$$

Where y is the vector of PTAs multiplied by 2, 1 is a vector of ones, μ is the bias, g=Mα is the vector of unobserved sums of marker effects (i.e., DGVs), M is the matrix of centered unphased genotypes with animals in rows and markers in columns, α is the vector of unobserved random allele substitution effects, and e is the vector of random residuals (i.e., polygenic errors). This regression model may be fitted by solving the following system of equations:

$$[ \begin{matrix}{1}^{T} 1 & {1}^{T} \\ 1 & I + {G}^{- 1} δ\end{matrix} ] [ \begin{matrix}\hat{μ} \\ \hat{g}\end{matrix} ] = [ \begin{matrix}{1}^{T} y \\ y\end{matrix} ]$$

Here G=ϕ⁻¹MM^{T} is the genomic relationship matrix (VanRaden, 2008), ϕ=σⱼ₌₁^{m}pⱼ (1−pⱼ) is the sum of genotype variances, δ=(1−r²)/r², and r²=0.95 is the assumed proportion of variance in PTAs explained by the N available markers. Marker effects may then be estimated by solving Strandén and Garrick's (2009) equation:

$$\hat{α} = {ϕ}^{- 1} {M}^{T} {G}^{- 1} \hat{g}$$

### Progeny Data

Sire-dam combinations in the reference data having genomically-tested progenies may be used for the validation of predicted distributions. Progeny genotypes may then be phased based on a reference haplotype library built from the reference data.

### Genetic Map

One may use empirical sex-specific recombination rates in one embodiment of the invention. To accommodate a particular SNP panel, marker coordinates in an original genetic map may first be updated from a relevant genome assembly. Then, recombination rates between consecutive markers in the map may be obtained using inverse-weight distance interpolation and the Kosambi mapping function. Recombination rates may be further scaled to preserve the estimated chromosome sizes in Morgans in the original genetic map.

### Analytical Approach for Estimating Gametic Moments

In an alternative embodiment of the invention, one may estimate gametic moments using an analytical approach as follows:

### Moments of the Distribution of a Random Variable

Under this approach, one may start by defining mean, covariance, correlation, coskewness and cokurtosis as follows (“Group 1 equations”):

$${μ}_{X} = E [ X ]$$

$${σ}_{X Y} = E [ ( X - {μ}_{X} ) ( Y - {μ}_{Y} ) ]$$

$${R}_{X Y} = E [ ( X - {μ}_{X} ) ( Y - {μ}_{Y} ) ] / \sqrt{{σ}_{X X} {σ}_{Y Y}}$$

$${S}_{X Y Z} = E [ ( X - {μ}_{X} ) ( Y - {μ}_{Y} ) ( Z - {μ}_{Z} ) ] / \sqrt{{σ}_{X X} {σ}_{Y Y} {σ}_{Z Z}}$$

$${K}_{X Y Z W} = E [ ( X - {μ}_{X} ) ( Y - {μ}_{Y} ) ( Z - {μ}_{Z} ) ( W - {μ}_{w} ) ] / \sqrt{{σ}_{X X} {σ}_{Y Y} {σ}_{Z Z} {σ}_{W W}}$$

where X, Y, Z and W represent random variables. Note that R, S, and K are scale-free statistics since they have the same units in the nominator and in the denominator. Also, for a probability density or mass function ƒ(x)=Pr(X=x), one has E[X]=∫_{-∞}^{∞}xƒ(x)dx and E[X]=Σᵢ₌₁ⁿxƒ(x) for continuous and discrete random variables respectively. In the case of estimations based on a sample of size n, ƒ(x)=1/n is assumed. From these definitions, the variance, skewness, and kurtosis of a random variable in this embodiment of the invention are (“Group 2 equations”):

$$VAR [ X ] = {σ}_{X}^{2} = {σ}_{X X}$$

$$SKEW [ X ] = {S}_{X}^{3} = {S}_{X X X}$$

$$KURT [ X ] = {K}_{X}^{4} = {K}_{XXXX}$$

Now, one can define the k^{th} raw, central, and standardized moments of a random variable respectively as (“Group 3 equations”):

$${μ}_{k}^{′} ( X ) = E [ {X}^{k} ]$$

$${μ}_{k} ( X ) = E [ {( X - {μ}_{X} )}^{k} ]$$

$$\frac{{μ}_{k} ( X )}{{σ}_{X}^{k}} = \frac{E [ {( X - {μ}_{X} )}^{k} ]}{{E [ {( X - {μ}_{X} )}^{2} ]}^{k / 2}}$$

Therefore, in this embodiment, the mean is the first raw moment, the variance is the second central moment, the skewness is the third standardized moment, and the kurtosis is the fourth standardized moment. In this embodiment of the invention, one may also write the covariance, correlation, coskewness, and cokurtosis as functions of raw moments as follows (“Group 4 equations”):

$${σ}_{X Y} = {μ}_{1}^{′} ( XY ) - {μ}_{1}^{′} ( X ) {μ}_{1}^{′} ( Y )$$

$${R}_{X Y} = \frac{1}{{σ}_{X} {σ}_{Y}} [ {μ}_{1}^{′} ( XY ) - {μ}_{1}^{′} ( X ) {μ}_{1}^{′} ( Y ) ]$$

$${S}_{X Y Z} = \frac{1}{{σ}_{X} {σ}_{Y} {σ}_{Z}} [ {μ}_{1}^{′} ( XYZ ) - {μ}_{1}^{′} ( X ) {μ}_{1}^{′} ( YZ ) - {μ}_{1}^{′} ( Y ) {μ}_{1}^{′} ( XZ ) - {μ}_{1}^{′} ( Z ) {μ}_{1}^{′} ( XY ) + 2 {μ}_{1}^{′} ( X ) {μ}_{1}^{′} ( Y ) {μ}_{1}^{′} ( Z ) ]$$

$${K}_{XYZW} = \frac{1}{{σ}_{X} {σ}_{Y} {σ}_{Z} {σ}_{W}} [ {μ}_{1}^{′} ( XYZW ) - {μ}_{1}^{′} ( XYZ ) {μ}_{1}^{′} ( W ) - {μ}_{1}^{′} ( XYW ) {μ}_{1}^{′} ( Z ) - {μ}_{1}^{′} ( XZW ) {μ}_{1}^{′} ( Y ) - {μ}_{1}^{′} ( YZW ) {μ}_{1}^{′} ( X ) + {μ}_{1}^{′} ( XY ) {μ}_{1}^{′} ( Z ) {μ}_{1}^{′} ( W ) + {μ}_{1}^{′} ( XZ ) {μ}_{1}^{′} ( Y ) {μ}_{1}^{′} ( W ) + {μ}_{1}^{′} ( XW ) {μ}_{1}^{′} ( Y ) {μ}_{1}^{′} ( Z ) + {μ}_{1}^{′} ( YZ ) {μ}_{1}^{′} ( X ) {μ}_{1}^{′} ( W ) + {μ}_{1}^{′} ( YW ) {μ}_{1}^{′} ( X ) {μ}_{1}^{′} ( Z ) + {μ}_{1}^{′} ( WZ ) {μ}_{1}^{′} ( X ) {μ}_{1}^{′} ( Z ) - 3 {μ}_{1}^{′} ( X ) {μ}_{1}^{′} ( Y ) {μ}_{1}^{′} ( Z ) {μ}_{1}^{′} ( W ) ]$$

Likewise, one may rewrite the variance, skewness, and kurtosis as (“Group 5 equations”):

$${σ}_{X}^{2} = {μ}_{2}^{′} ( X ) - {{μ}_{1}^{′} ( X )}^{2}$$

$${S}_{X}^{3} = [ {μ}_{3}^{′} ( X ) - 3 {μ}_{1}^{′} ( X ) {μ}_{2}^{′} ( X ) + 2 {{μ}_{1}^{′} ( X )}^{3} ] / {σ}_{X}^{3}$$

$${K}_{X}^{4} = [ {μ}_{4}^{′} ( X ) - 4 {μ}_{1}^{′} ( X ) {μ}_{3}^{′} ( X ) + 6 {{μ}_{1}^{′} ( X )}^{2} {μ}_{2}^{′} ( X ) - 3 {{μ}_{1}^{′} ( X )}^{4} ] / {σ}_{X}^{4}$$

It is further possible to express raw moments as functions of the parameters of a distribution by using the moment generating function (MGF) (“Group 6 equations”):

$${M}_{t} ( X ) = E [ {e}^{tX} ] = 1 + t {μ}_{1}^{′} ( X ) + \frac{{t}^{2} {μ}_{2}^{′} ( X )}{2 !} + \frac{{t}^{3} {μ}_{3}^{′} ( X )}{3 !} + \frac{{t}^{4} {μ}_{4}^{′} ( X )}{4 !} + …$$

The MGF is useful because setting the k^{th}-order derivative of Mₜ(X) in respect to t and evaluating it at t=0 is a way of finding an expression for μ′ₖ(X) that is dependent on the parameters of the function that generates the random variable X. For example, for the Bernoulli distribution that describes a random variable assuming value 1 with probability π and value 0 with probability 1−π, the probability mass function is (“Group 7 equations”):

$$P r ( X = x \vert π ) = {{π}^{x} ( 1 - π )}^{1 - x}$$

Evaluating E[e^{tX}]=Σe^{tx}ƒ(x) gives the following MGF (“Group 8 equation”):

$${M}_{t} ( X ) = 1 - π + π {e}^{t}$$

For which all raw moments are then found to be μ′ₖ(X)=π. Substituting these raw moments in the Group 5 equations, and rearranging the resulting expressions, one gets (“Group 9 equations”):

$${μ}_{X} = π$$

$${σ}_{X}^{2} = π ( 1 - π )$$

$${S}_{X}^{3} = ( 1 - 2 π ) / \sqrt{π ( 1 - π )}$$

$${K}_{X}^{4} = 3 + [ 1 - 6 π ( 1 - π ) ] / [ π ( 1 - π ) ]$$

If the random variable under investigation results from the sum of N other random variables, one may further express its mean, variance, skewness, and kurtosis as (“Group 10 equations”):

$${μ}_{t} = {\sum }_{j = 1}^{N} {μ}_{j}$$

$${σ}_{t}^{2} = {\sum }_{j = 1}^{N} {σ}_{j}^{2} + 2 {\sum }_{j = 1}^{N} {\sum }_{k > j}^{N} {σ}_{j} {σ}_{k} {R}_{j k}$$

$${S}_{t}^{3} = \frac{1}{{σ}_{t}^{3}} [ {\sum }_{j = 1}^{N} {S}_{j}^{3} {σ}_{j}^{3} + 3 {\sum }_{j = 1}^{N} {\sum }_{k > j}^{N} ( {σ}_{j}^{2} {σ}_{k} {S}_{jjk} + {σ}_{j} {σ}_{k}^{2} {S}_{j k k} ) + 6 {\sum }_{j = 1}^{N} {\sum }_{k > j}^{N} {\sum }_{l > k}^{N} {σ}_{j} {σ}_{k} {σ}_{l} {S}_{j k l} ]$$

$${K}_{t}^{4} = \frac{1}{{σ}_{t}^{4}} [ \text{} {\sum }_{j = 1}^{N} {K}_{j}^{4} {σ}_{j}^{4} + 2 {\sum }_{j = 1}^{N} {\sum }_{k > j}^{N} ( 2 {σ}_{j}^{3} {σ}_{k} {K}_{j j j k} + 3 {σ}_{j}^{2} {σ}_{k}^{2} {K}_{j j k k} + 2 {σ}_{j} {σ}_{k}^{3} {K}_{jkkk} ) + 12 {\sum }_{j = 1}^{N} {\sum }_{k > j}^{N} {\sum }_{l > k}^{N} ( {σ}_{j}^{2} {σ}_{k} {σ}_{l} {K}_{j j k l} + {σ}_{j} {σ}_{k}^{2} {σ}_{l} {K}_{j k k l} + {σ}_{j} {σ}_{k} {σ}_{l}^{2} {K}_{j k l l} ) + 24 {\sum }_{j = 1}^{N} {\sum }_{k > j}^{N} {\sum }_{l > k}^{N} {\sum }_{m > l}^{N} {σ}_{j} {σ}_{k} {σ}_{l} {σ}_{m} {K}_{j k l m} ]$$

In this embodiment, using these equations, one may then find the moments of the distribution of breeding values in gametes and progenies.

### Moments of the Distribution of Gametes

With respect to a single SNP site, sampling a B allele is essentially a Bernoulli trial with probability of success n assuming values 0, 0.5 and 1 for genotypes AA, AB, and BB, respectively. The moments of the Bernoulli distribution are described in the previous section. However, for haplotypes, one centers the indicator variable of each marker on its allele frequency and then multiplies it by the substitution effect. This causes the MGF to change to (“Group 11 equation”):

$${M}_{t} ( X ) = {e}^{t ( 0 - p ) α} ( 1 - π ) + {e}^{t ( 1 - p ) α} π$$

Setting the k^{th}-order derivative of Mₜ(X) in respect to t and evaluating it at t=0 one gets the k^{th} raw moment as (“Group 12 equation”):

$${μ}_{k}^{′} ( X ) = {( 1 - π ) [ ( 0 - p ) α ]}^{k} + {π [ ( 1 - p ) α ]}^{k}$$

Therefore, the mean, variance, skewness, and kurtosis become (π−p)α, π(1−π)α², (1−2π)/\sqrt{π(1−π)} and 3+[1−6π(1−π)]/[π(1−π)], respectively. Note that only the mean and variance are affected. By replacing π with values 0 (AA), 0.5 (AB) or 1 (BB), one finds that the two homozygote classes have zero variance and, consequently, undefined skewness and kurtosis. For the heterozygote class one finds variance=0.25α², skewness=0 and kurtosis=1. This implies that only heterozygous loci can contribute to the variance, skewness, and kurtosis of the distribution of gametes, despite homozygous loci still contributing to the mean. To generalize for multiple loci in a chromosome, one now needs to find expressions for the correlation, coskewness and cokurtosis terms and substitute them in the Group 10 equations. Since these statistics are scale-free, computing them based on the raw random variables yields the same results as for centered and scaled random variables. Therefore, taking advantage of the observation in the Group 8 equations that all raw moments of a Bernoulli trial without scaling or centering are μ′ₖ(X)=π, one can substitute raw moments in the Group 4 equations to find (“Group 13 equations”):

$${R}_{j k} = ( {π}_{j k} - {π}_{j} {π}_{k} ) / \sqrt{{π}_{j} ( 1 - {π}_{j} ) {π}_{k} ( 1 - {π}_{k} )} \\ {S}_{jkl} = ( {π}_{jkl} - {π}_{j} {π}_{kl} - {π}_{k} {π}_{jl} - {π}_{l} {π}_{jk} + 2 {π}_{j} {π}_{k} {π}_{l} ) / \sqrt{{π}_{j} ( 1 - {π}_{j} ) {π}_{k} ( 1 - {π}_{k} ) {π}_{l} ( 1 - {π}_{l} )} \\ {K}_{jklm} = ( {π}_{jklm} - {π}_{j} {π}_{klm} - {π}_{k} {π}_{jlm} - {π}_{l} {π}_{jkm} - {π}_{m} {π}_{jkl} + {π}_{j} {π}_{k} {π}_{lm} + {π}_{j} {π}_{l} {π}_{km} + {π}_{j} {π}_{m} {π}_{kl} + {π}_{k} {π}_{l} {π}_{jm} + {π}_{k} {π}_{m} {π}_{jl} + {π}_{l} {π}_{m} {π}_{jk} - 3 {π}_{j} {π}_{k} {π}_{l} {π}_{m} ) \text{} / \sqrt{{π}_{j} ( 1 - {π}_{j} ) {π}_{k} ( 1 - {π}_{k} ) {π}_{l} ( 1 - {π}_{l} ) {π}_{m} ( 1 - {π}_{m} )}$$

Where any π parameter here is the joint probability of success, i.e., the probability of observing only B alleles in the sampled haplotype across the respective subscripted loci. As previously discussed, all π parameters with single subscript are equal to 0.5 for heterozygous loci, so one can simplify the Group 13 equations to (“Group 14 equations”):

$${R}_{jk} = 4 {π}_{jk} - 1 \\ {S}_{jkl} = 8 {π}_{jkl} - 4 ( {π}_{kl} + {π}_{jl} + {π}_{jk} ) + 2 \\ {K}_{jklm} = 16 {π}_{jklm} - 8 ( {π}_{klm} + {π}_{jlm} + {π}_{jkm} + {π}_{jkl} ) + 4 ( {π}_{lm} + {π}_{km} + {π}_{kl} + {π}_{jm} + {π}_{jl} + {π}_{jk} ) - 3$$

One may then find the remaining n parameters. Starting with the simplest case, for two loci j and k, the parameter πⱼₖ will be a function of the recombination rate and the linkage phase. If the two haplotypes represented in binary code are 01 and 10, then sampling a 11 gamete will depend on the probability ρⱼₖ of a crossing-over event and then on the probability of sampling the recombinant haplotype 11, which will be ½. Therefore, the probability of sampling a 11 gamete is πⱼₖ=ρⱼₖ/2. Now, if the two haplotypes were 00 and 11, then the probability of obtaining a 11 gamete would be ½ times the probability of not getting a recombinant haplotype, that is πⱼₖ=(1−ρⱼₖ)/2. Following this logic, one may state all possible recombination and sampling events for 3 and 4 loci to find all expressions of π parameters. One may represent these using matrix notation as (“Group 15 equations”):

$$[ \begin{matrix}{{π}_{jk}}_{( 11 / 00 )} \\ {π}_{j {k}_{( 10 / 01 )}}\end{matrix} ] = \frac{1}{2} [ \begin{matrix}1 & - 1 \\ 0 & 1\end{matrix} ] [ \begin{matrix}1 \\ {ρ}_{jk}\end{matrix} ] \\ [ \begin{matrix}{{π}_{jkl}}_{( 111 / 000 )} \\ {{π}_{jkl}}_{( 110 / 001 )} \\ {{π}_{jkl}}_{( 100 / 011 )} \\ {{π}_{jkl}}_{( 101 / 010 )}\end{matrix} ] = [ \begin{matrix}1 & - 1 & - 1 & 1 \\ 0 & 1 & 0 & - 1 \\ 0 & 0 & 1 & - 1 \\ 0 & 0 & 0 & 1\end{matrix} ] [ \begin{matrix}1 \\ {ρ}_{kl} \\ {ρ}_{jk} \\ {ρ}_{jk} {ρ}_{kl}\end{matrix} ] \\ [ \begin{matrix}{{π}_{jklm}}_{( 11 \; 11 / 0000 )} \\ {{π}_{jklm}}_{( 1110 / 0001 )} \\ {{π}_{jklm}}_{( 1100 / 0011 )} \\ {π}_{{jklm}_{( 1101 / 0010 )}} \\ {π}_{{jklm}_{( 1000 / 0111 )}} \\ {π}_{{jklm}_{( 1001 / 0110 )}} \\ {π}_{{jklm}_{( 1011 / 0100 )}} \\ {π}_{{jklm}_{( 1010 / 0101 )}}\end{matrix} ] = [ \begin{matrix}1 & - 1 & - 1 & 1 & - 1 & 1 & 1 & - 1 \\ 0 & 1 & 0 & - 1 & 0 & - 1 & 0 & 1 \\ 0 & 0 & 1 & - 1 & 0 & 0 & - 1 & 1 \\ 0 & 0 & 0 & 1 & 0 & 0 & 0 & - 1 \\ 0 & 0 & 0 & 0 & 1 & - 1 & - 1 & 1 \\ 0 & 0 & 0 & 0 & 0 & 1 & 0 & - 1 \\ 0 & 0 & 0 & 0 & 0 & 0 & 1 & - 1 \\ 0 & 0 & 0 & 0 & 0 & 0 & 0 & 1\end{matrix} ] [ \begin{matrix}1 \\ {ρ}_{lm} \\ {ρ}_{kl} \\ {ρ}_{kl} {ρ}_{lm} \\ {ρ}_{jk} \\ {ρ}_{jk} {ρ}_{lm} \\ {ρ}_{jk} {ρ}_{kl} \\ {ρ}_{jk} {ρ}_{kl} {ρ}_{lm}\end{matrix} ]$$

The upper triangular matrix of coefficients may be conveniently built from successive Kronecker products of the square matrix:

$$[ \begin{matrix}1 & - 1 \\ 0 & 1\end{matrix} ]$$

Values for the required ρ parameters may be obtained from estimated genetic maps. The recombination rates required for pairs of non-consecutive markers, or for segments containing 3 or 4 loci, may be further approximated under the assumption of additivity, for example, using Haldane's or Kosambi's mapping functions. From the expressions above, and assuming that the recombination rate of a locus with itself is ρᵢⱼ=0, it is further useful to note the following identities: πⱼⱼⱼₖ=πⱼₖₖₖ=πⱼⱼₖₖ=πⱼⱼₖ=πⱼₖ, πⱼⱼₖₗ=πⱼₖₖₗ=πⱼₖₗₗ=πⱼₖₗ and πⱼⱼⱼⱼ=πⱼⱼⱼ=πⱼⱼ=πⱼ=0.5. Using these identities and substituting the π parameters from the Group 15 equations into the expressions in the Group 14 equations, with further simplification of the expressions, we find (“Group 16 equations”):

$${R}_{jk} = {L}_{jk} ( 1 - 2 {ρ}_{jk} ) \\ {S}_{jkl} = {R}_{jk} {R}_{kl} - {R}_{jl} \\ {K}_{jklm} = ( {S}_{jkl} + {R}_{jk} ) {R}_{lm} - {S}_{jkm}$$

where Lⱼₖ is a linkage phase indicator assuming value 1 if the two loci have their B allele in the same haplotype and −1 otherwise. Since ρⱼⱼ=0, we have Rⱼⱼ=1, Sⱼⱼⱼ=Sⱼⱼₖ=Sⱼₖₖ=0, Kⱼⱼⱼⱼ=Kⱼⱼₖₖ=1, Kⱼⱼⱼₖ=Kⱼₖₖₖ=Kⱼₖᵤₗ=Rⱼₖ, Kⱼⱼₖₗ=Rₖₗ and Kⱼₖₖₗ=Rⱼₗ. Also, a useful alternative formulation for the correlation terms uses genetic distances in replacement of recombination rates (“Group 17 equation”):

$${R}_{jk} = {L}_{jk} ( 1 - \frac{min ( c {M}_{jk} , 50 )}{50} )$$

where cMⱼₖ is the distance between markers in centimorgans. One may now return to the Group 10 equations to re-write the moments of a chromosome as functions of allele substitution effects and linkage correlations as follows (“Group 18 equations”):

$${μ}_{chr} = \frac{1}{2} {\sum }_{j = 1}^{{N}_{chr}} {m}_{ij} {α}_{j} \\ {σ}_{chr}^{2} = \frac{1}{4} [ {\sum }_{j = 1}^{{N}_{het}} {α}_{j}^{2} + 2 {\sum }_{j = 1}^{{N}_{het}} {\sum }_{k > j}^{{N}_{het}} {R}_{jk} {α}_{j} {α}_{k} ] \\ {S}_{chr}^{3} = \frac{3}{4} [ {\sum }_{j = 1}^{{N}_{het}} {\sum }_{k > j}^{{N}_{het}} {\sum }_{l > k}^{{N}_{het}} ( {R}_{jk} {R}_{kl} - {R}_{jl} ) {α}_{j} {α}_{k} {α}_{l} ] / {σ}_{chr}^{3} \\ {K}_{chr}^{4} = [ \frac{1}{16} {\sum }_{j = 1}^{{N}_{het}} {α}_{j}^{4} + \frac{1}{8} {\sum }_{j = 1}^{{N}_{het}} {\sum }_{k > j}^{{N}_{het}} {R}_{jk} ( {α}_{j}^{3} {α}_{k} + 3 {α}_{j}^{2} {α}_{k}^{2} + {α}_{j} {α}_{k}^{3} ) + \frac{3}{4} {\sum }_{j = 1}^{{N}_{het}} {\sum }_{k > j}^{{N}_{het}} {\sum }_{l > k}^{{N}_{het}} ( {α}_{j}^{2} {α}_{k} {α}_{l} {R}_{kl} + {α}_{j} {α}_{k}^{2} {α}_{l} {R}_{jl} + {α}_{j} {α}_{k} {α}_{l}^{2} {R}_{jk} ) + 3 {\sum }_{j = 1}^{{N}_{het}} {\sum }_{k > j}^{{N}_{het}} {\sum }_{l > k}^{{N}_{het}} {\sum }_{m > l}^{{N}_{het}} {α}_{j} {α}_{k} {α}_{l} {α}_{m} ( {R}_{jk} {R}_{kl} {R}_{lm} + {R}_{jk} {R}_{lm} - {R}_{jk} {R}_{km} - {R}_{jl} {R}_{lm} + {R}_{jm} ) ] / {σ}_{chr}^{4}$$

Where N_{chr} and Nₕₑₜ are the number of markers and the number of heterozygous loci on the target chromosome, respectively. The skewness and kurtosis formulae above imply that fitness to a normal distribution (i.e., S_{chr}³=0 and K_{chr}⁴=3) with mean μ_{chr} and variance σ_{chr}² will depend on the distribution of allele substitution effects, haplotype linkage phase and recombination rates between heterozygous loci. It is also important to note that these four moments do not inform about multimodality, so there is no guarantee that the distribution of gametes within chromosomes will be bell-shaped. Assuming independence of chromosomes, the moments for the whole genome may be estimated from the individual chromosome distributions as (“Group 19 equations”):

$${μ}_{gamete} = {\sum }_{chr = 1}^{{N}_{chr}} {μ}_{chr} \\ {σ}_{gamete}^{2} = {\sum }_{chr = 1}^{{N}_{chr}} {σ}_{chr}^{2} \\ {S}_{gamete}^{3} = \frac{1}{{σ}_{g amete}^{3}} {\sum }_{chr = 1}^{{N}_{chr}} {S}_{chr}^{3} {σ}_{chr}^{3} \\ {K}_{gamete}^{4} = \frac{1}{{σ}_{gamete}^{4}} [ {\sum }_{chr = 1}^{{N}_{chr}} {K}_{chr}^{4} {σ}_{chr}^{4} + 6 {\sum }_{chr = 1}^{{N}_{chr}} {\sum }_{{chr}^{′} > chr}^{{N}_{chr}} {σ}_{chr}^{2} {σ}_{{chr}^{′}}^{2} ]$$

Since the gametic distribution for the whole genome results from the sum of several independent chromosome distributions, one should expect it to converge to a unimodal distribution. However, fitness to a normal distribution will still depend on convergence of skewness to 0 and kurtosis to 3 in the formulae above, which is not guaranteed.

### Computational Complexity

Using the analytical approach embodiment of the invention, the computational burden of gametic moments lies on the large number of terms to compute per animal, especially for kurtosis. The number of terms under summations for each chromosome is given by the sum of binomial coefficients:

$${\sum }_{chr = 1}^{{N}_{chr}} \frac{{N}_{chr} !}{b ! ( {N}_{chr} - b ) !}$$

Where b is the number of nested summations. By way of example only, for the filtered set of 56,034 SNPs in the ST/GVI-VM2 genotyping array, the maximum number of terms to compute under every single, double, and triple summation is approximately 56 thousand, 59 million and 45 billion, respectively. However, heterozygosity will limit the number of terms to be computed. For an animal with heterozygosity of 20%, at most 11 thousand, 2.3 million and 359 million terms would need to be computed under single, double, and triple summations, respectively, when genetic distances over 50 cM are not ignored. Using standard and efficient C++ libraries, one finds that these terms may be computed under nested for loops with O(N_{chr}^{b}) complexity. For bovine chromosome 1 for example, the variance, skewness, and kurtosis cost an average of 5 milliseconds, 1 second and 3 minutes per animal to be computed, respectively. It is therefore clear that the variance and skewness parameters are feasible to implement for millions of animals using parallel computations, whereas the kurtosis may be difficult to maintain in large genotype cohorts. In contrast, as shown below, the sampling algorithm to bovine chromosome 1 across 45 simulated traits (9 models×5 replicates) using a sample size of 1,000, 10,000 and 100,000 completes calculations respectively under 0.5, 5 and 50 seconds per animal.

### Example 1

A set of 11,815 bulls and 1,558 cows genotyped for 63,836 SNP markers (ST/GVI-VM2 genotyping array) was used. Since only uniquely mapped autosomal markers with call rate >95%, heterozygosity <70% and minor allele frequency >1% were considered for analysis, the initial panel was reduced to 56,034 SNPs. Genotypes were then phased using the population-based method implemented in Eagle v2.4.1 (Loh et al., 2016). The mean phasing confidence estimated by a 5-fold cross-validation was 99.51%, with a minimum of 96.54% and a maximum of 99.96%. Allele substitution effects for milk, fat and protein were obtained by regressing PTAs onto marker genotypes. Marker effects were then estimated by solving Stranden and Garrick's (2009) equation. The mean±standard deviation and the range of milk, fat and protein PTAs were 887±674 (−2004 to 3588), 69±34 (−61 to 171) and 41±20 (−50 to 103), respectively. The average reliability across traits was 85%.

A total of 122 sire-dam combinations in the reference data had at least 10 genomically-tested progenies each and were therefore used for the validation of predicted distributions. These families comprised 44 bulls, 103 cows and 1,563 offspring (727 males and 836 females). The average number of offspring per family was 13, and the maximum was 42. Progeny genotypes were phased based on the reference haplotype library built from the previously described 13,373 animals, and had phasing confidences ranging from 97.40% and 99.92%, with a mean of 99.61%.

Previously published sex-specific recombination rates estimated for Holstein (Ma et al., 2015; Shen et al., 2018) were used. To accommodate the SNP panel, marker coordinates in the original genetic map were first updated from the UMD v3.1.1 (Zimin et al., 2017) to the ARS-UCDv1.2 (Rosen et al., 2020) genome assembly. Then, recombination rates between consecutive markers in our map were obtained using inverse-weight distance interpolation and the Kosambi mapping function. Recombination rates were further scaled to preserve the estimated chromosome sizes in Morgans in the original genetic map.

All computer code required for the analyses described in this Example were developed in-house and implemented using a combination of Rcpp (Eddelbuettel & François, 2011) in R v4.2.2 (R Core Team, 2023) and standard C++ libraries. Gamete statistics were stored in a Microsoft® SQL Server database. Distribution approximations based on cumulants were performed with the PDQutils package in R (Pav, 2017). Graphical visualizations were developed with ggplot2 (Wickham, 2016). All analyses were carried out in a computer node with 256 CPUs (AMD EPYC 7742 64-Core processors) and 504 GB of RAM running Ubuntu 22.04 LTS.

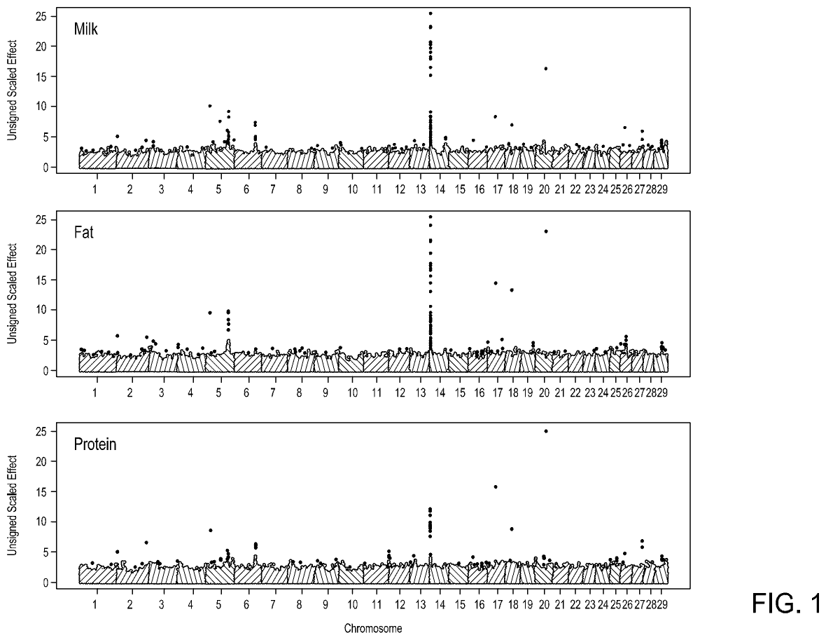

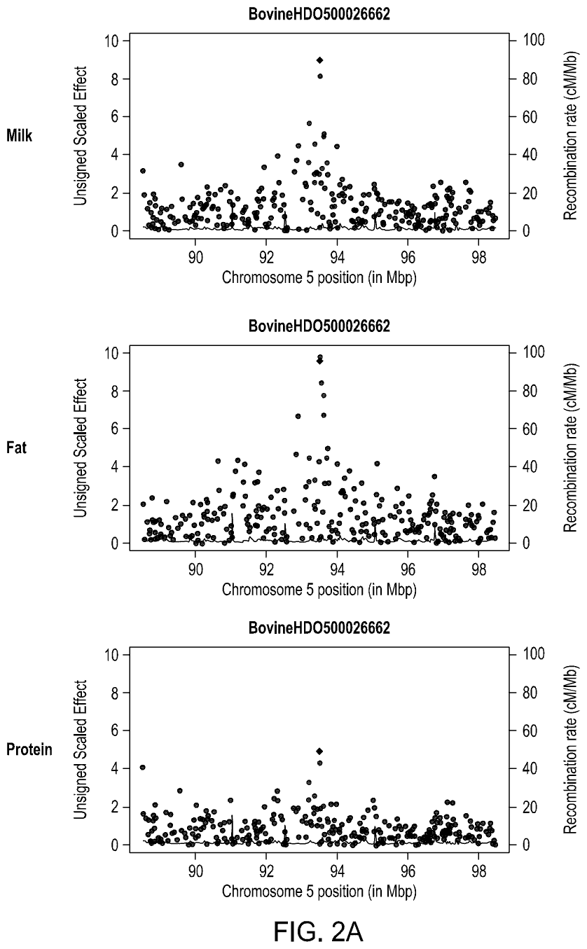

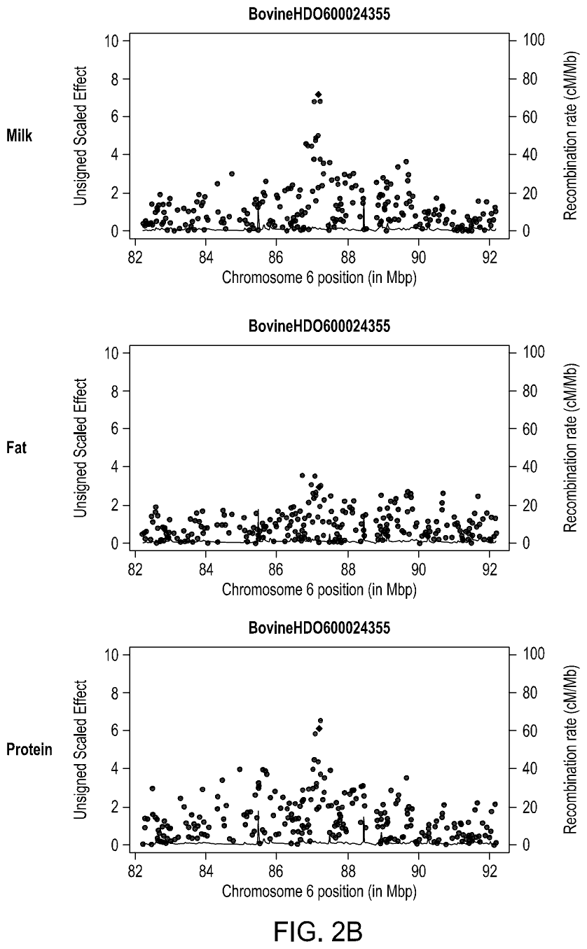

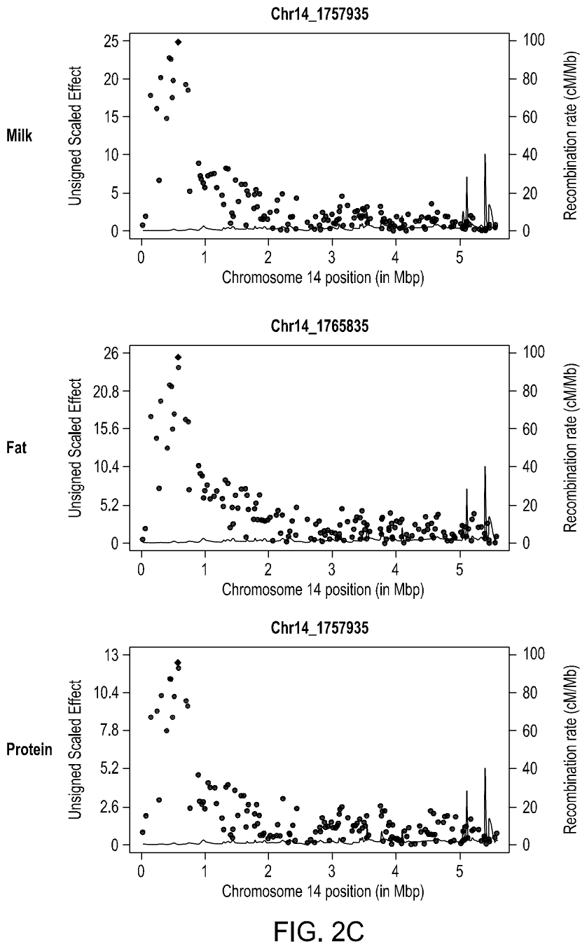

The solution of marker effects replicated known QTLs for milk, fat, and protein in Holstein cattle (FIG. 1), more noticeably on chromosomes 5, 6 and 14 (FIGS. 2A to 2C, respectively). These effects were applied to haplotype windows of 100 markers for the invention assuming a number of independent chromosomal segments of Mₑ=562 for a population with effective size Nₑ=100 and autosomal genome length of L=26 Morgans (L=27 for males and L=25 for females). For an improved smoothing effect in the visualization of haplotypes, windows were allowed to overlap by a step size of 25 markers (¼ of the window size).

FIG. 1 shows the allele substitution effect solutions for milk, fat, and protein yield in Holstein cattle. For clarity, the effects are displayed as absolute standard deviates from the mean effect size of each trait.

FIGS. 2A to 2C show regional plots of major QTLs on autosomes 5, 6 and 14, respectively, affecting milk, fat, and protein yield in Holstein cattle.

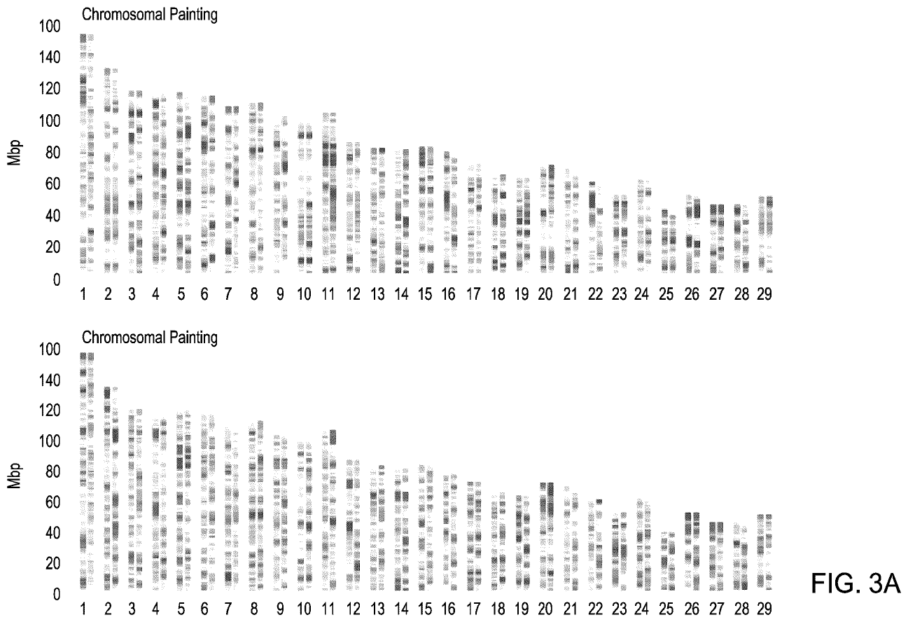

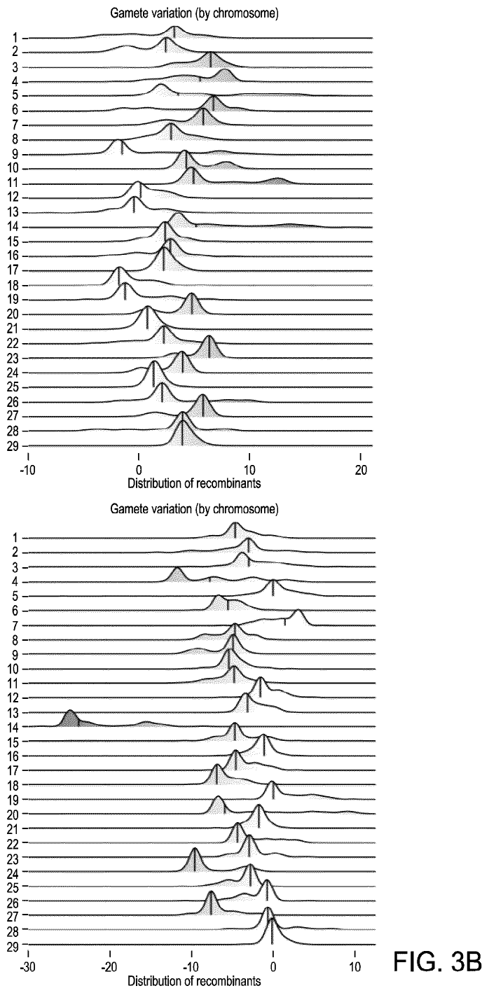

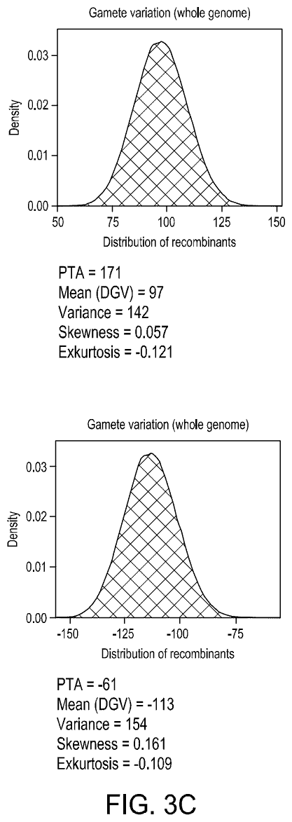

To illustrate the visual differentiation of DGVs of chromosomal segments, the animals with the lowest and the highest PTAs for fat yield were first selected (FIG. 3A). (Note that the chromosomal painting and gamete variations for the animal with the highest PTA for fat yield are shown on top in each of FIGS. 3A to 3C, and the chromosomal painting and gamete variations for the animal with the lowest PTA for fat yield are shown on the bottom in each of FIGS. 3A to 3C.) As expected, the highest PTA animal concentrated more positive than negative segments, whereas negative segments predominated in the haplotypes of the lowest PTA animal. However, both animals presented haplotypes with positive and negative net DGV, and each haplotype presented both positive and negative segments regardless of the sign of their net DGV. Simulation of gametes based on these painted haplotypes revealed distributions of recombinants with a wide range of shapes departing from normality, including heavy-tailed, skewed distributions (FIG. 3B). Multimodal distributions were also observed for some chromosome pairs, which may reflect the influence of segregating large effects segments on the genetic merit of recombinant haplotypes. The highest PTA animal for fat yield produced mostly positive distributions for net DGV recombinants across chromosome pairs, whereas the lowest PTA animal mostly produced negative net DGV recombinants. After aggregation of chromosome-specific distributions for the whole genome, the distribution of gametes became unimodal, albeit still slightly skewed and with heavier tails than a normal distribution (FIG. 3C).

FIG. 3A shows the visual differentiation of DGVs of chromosomal segments of two animals with extreme merits for fat yield. Visually differentiated phased genotypes partitioned into segments of 100 markers (“chromosomal painting”) are shown in FIG. 3A. Red segments make negative contributions, whereas blue segments contribute positively to the DGV. The number above each haplotype indicates the haplotype-specific weight to the DGV. The density plots in FIG. 3B show the gamete variation resulting from recombination of paternal haplotypes shown in the chromosomal painting, and the density plots in FIG. 3C aggregate that data for the whole genome. Although major QTLs yielded multimodal distributions for some chromosome pairs, the overall genome-wide distribution of gametes was unimodal in FIG. 3C. In both cases the distribution of gametes was not normal. The negative excess kurtosis indicated heavier tails than a typical normal distribution, and the positive skewness further suggested a distribution that was slightly skewed to the right. Note that in FIG. 3, PTA=DGV+(Bias+Polygenic Error)/2, where Bias=147.495 (intercept of regression) and Polygenic Error is the individual residual effect from PTA regression onto markers.

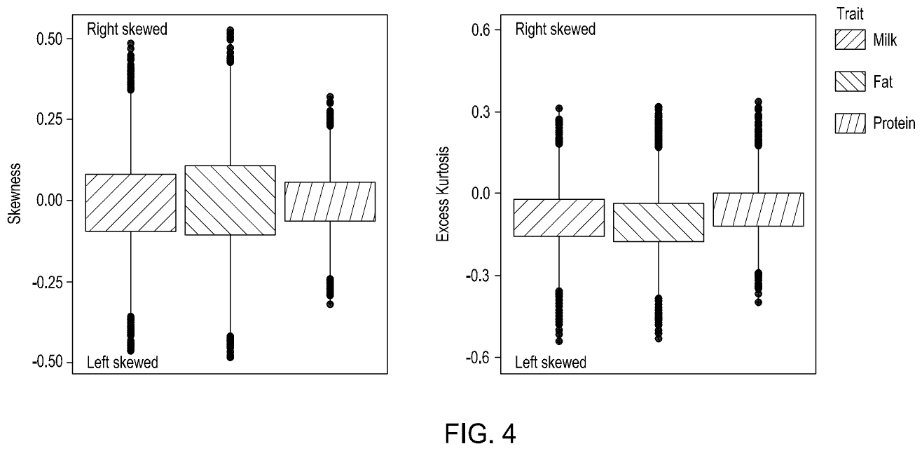

Next, the variability of gamete distribution shapes across animals was characterized by investigating the distribution of skewness and excess kurtosis estimates in the genotyped population (FIG. 4). Milk and fat presented more variation in skewnesss values and overall lower excess kurtosis as compared to protein, most likely reflecting the largest QTL effects found for these traits. All traits had the third quartile (75% of the data) of excess kurtosis below zero, indicating that most animals had gametic distributions with heavier tails than a typical normal distribution. Positive excess kurtosis was also found for the upper quartile of the data, indicating that some animals may further have leptokurtic distributions that are less prone to the production of outlier gametes.

FIG. 4 shows the distribution of gametic skewness and (excess) kurtosis for milk, fat and protein.

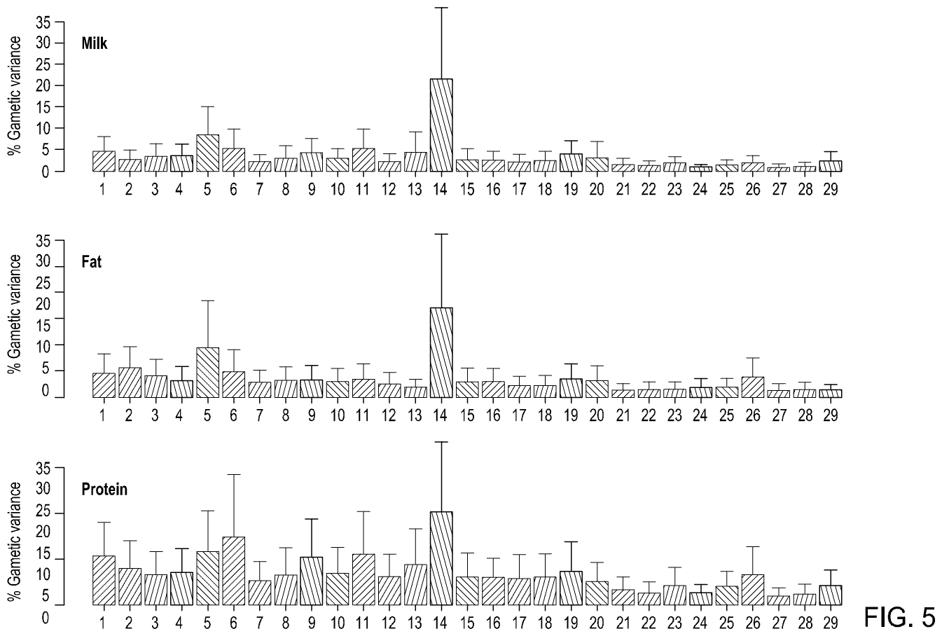

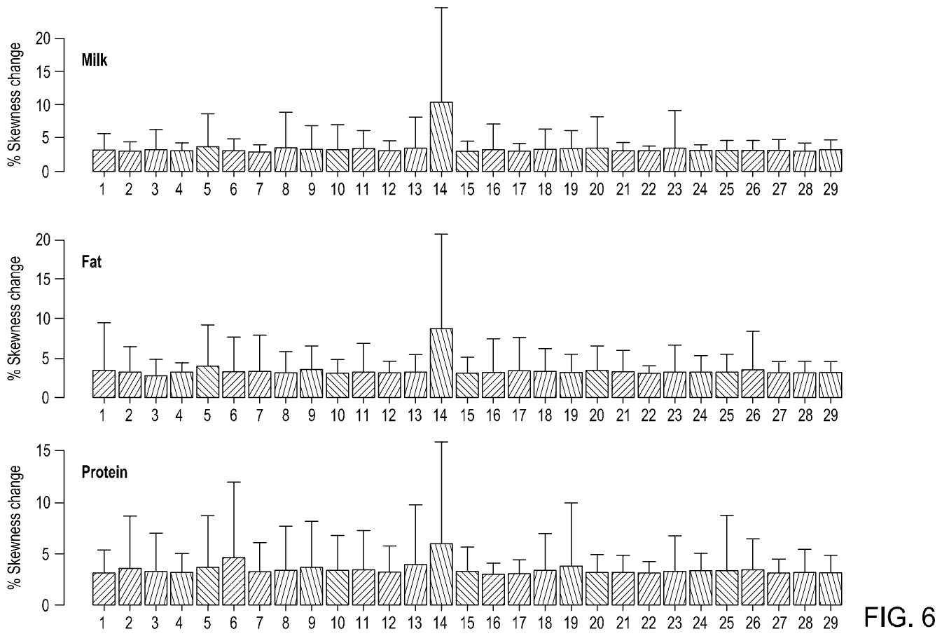

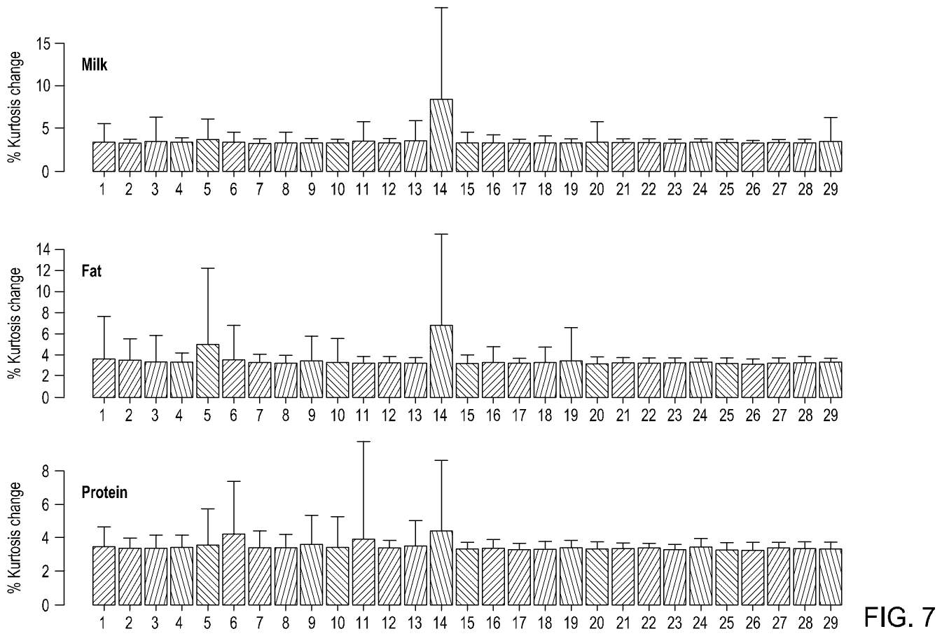

To further understand the source of variation in gametic moments, the variance, skewness and kurtosis of the aggregated distributions of each animal were regressed on their genomic inbreeding as measured by the diagonal elements of the GRM. Although inbreeding had a significant negative effect on gametic variance, it could only explain 2.7% (p=1.92×10−103), 4.8% (p=2.41×10−188) and 5.2% (p=3.31×10−204) of the differences across animals for milk, fat and protein yield respectively. In contrast, inbreeding had no significant impact (p>0.05) on skewness or kurtosis. Therefore, most of the differences in the shape of the distribution of gametes across animals may be explained by other factors such as recombination rates, gametic phase or specific haplotypic configurations of segmental DGVs, especially influenced by the presence or absence of major QTLs. To test that hypothesis, the data were reanalyzed by suppressing one chromosome at a time to measure their impact on the aggregate value of gametic moments. For the variance, this impact was measured by simply dividing the chromosome-specific variance by the total variance, as proxy for the proportion of gametic variance explained by a given chromosome (FIG. 5). For skewness (FIG. 6) and kurtosis (FIG. 7), the ratio between the values including and excluding each chromosome was calculated, and then each ratio was scaled by the sum of ratios across all autosomes. Consistent impacts of chromosomes 5, 6 and 14 were found on all gametic moments, indicating that major QTLs can significantly affect the shape and variability of gamete distributions.

FIG. 5 shows the mean (bars) and standard deviations (whiskers) for the percentage of total gametic variance explained by recombinant haplotypes at specific chromosome pairs.

FIG. 6 shows the mean (bars) and standard deviations (whiskers) for the relative change in gametic skewness caused by recombinant haplotypes at specific chromosome pairs.

FIG. 7 shows the mean (bars) and standard deviations (whiskers) for the relative change in gametic excess kurtosis caused by recombinant haplotypes at specific chromosome pairs.

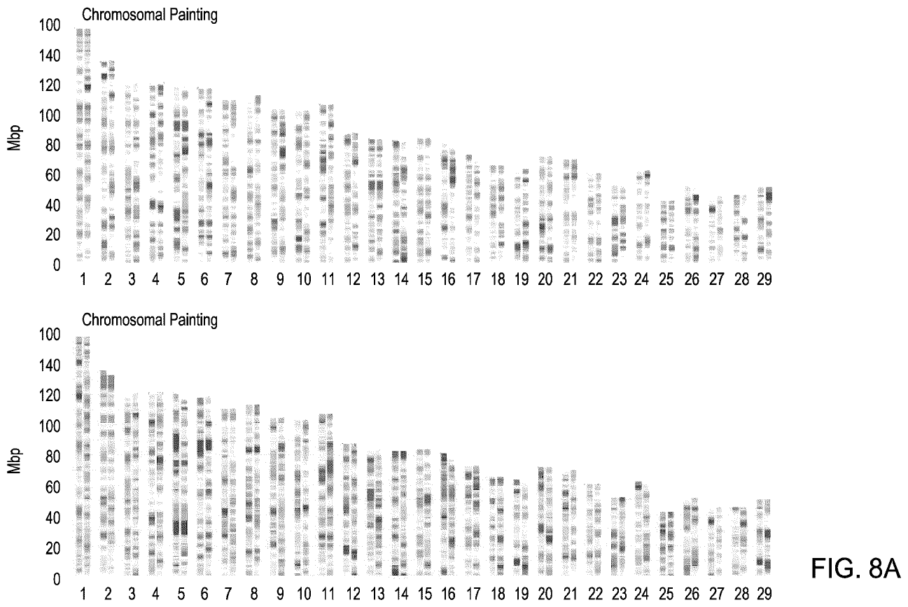

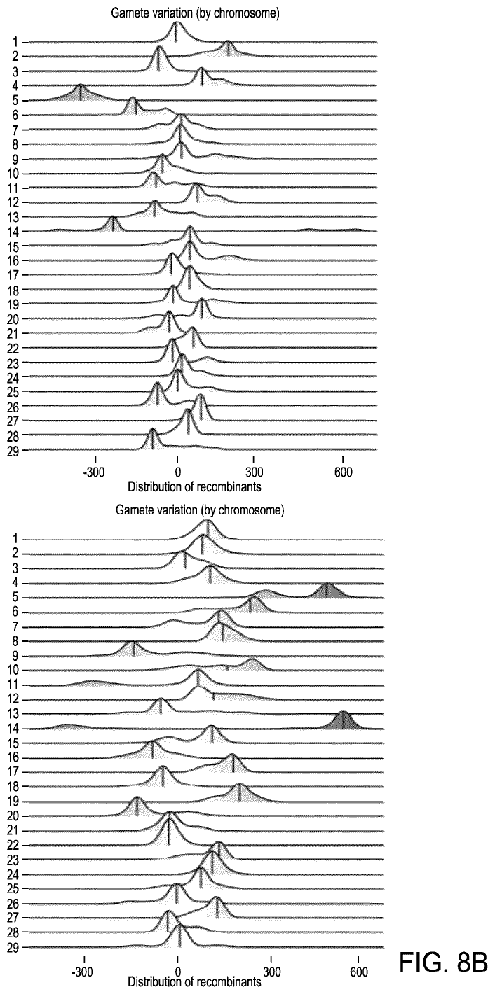

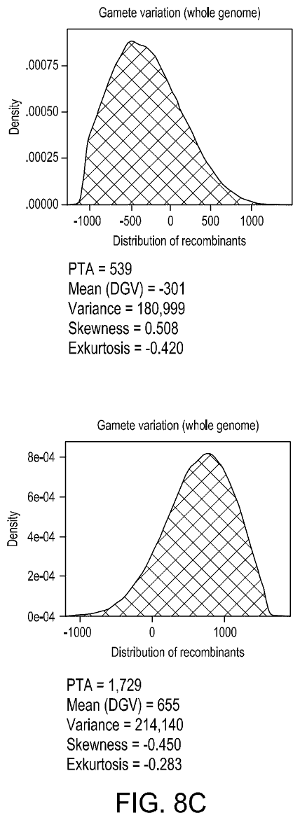

To inspect the influence of QTLs on distribution shapes more closely, DGVs of chromosomal segments were visually differentiated for two animals with opposing skewness values for milk yield (FIG. 8A). (Note that the chromosomal painting and gamete variations for the animal with positive skewness for milk yield are shown on top in each of FIGS. 8A to 8C, and the chromosomal painting and gamete variations for the animal with negative skewness for milk yield are shown on the bottom in each of FIGS. 8A to 8C.) Bimodal distributions for recombinant haplotypes with very large distances between modes were observed on chromosome. Removing chromosome 14 from the analysis caused the distribution of gametes to closely converge to a normal distribution, with skewness and excess kurtosis nearing zero. The DGVs of recombinants of the chromosome 14 pair further accounted for 55% and 67% for the animals with right- and left-skewed gamete distributions respectively. This reinforces that large QTL effects are key drivers of gametic variation and deviations of the gametic distribution from the shape of a normal distribution. By performing an analytical derivation of the moments of gametic distributions as described above, this hypothesis was further supported from a theoretical standpoint. Furthermore, simulation of different distributions of SNP effects showed that skewness has more variation and average kurtosis is lowered when shrinkage on marker effects is alleviated by using variable selection methods while solving for allele substitution effects (see Example 2).

In FIG. 8A, DGVs of chromosomal segments were visually differentiated for two animals with opposing skewness for milk yield. FIGS. 8A, 8B and 8C are as described for FIGS. 3A, 3B and 3C. Referring to FIG. 8B, large negative and positive contributions to the gametic DGV are observed for chromosomes 5 and 14, apart from extreme bimodal distributions for chromosome 14. Note that in FIG. 8, PTA=DGV+ (Bias+Polygenic error)/2, where Bias=1,960.819 (intercept of regression) and Polygenic Error is the individual residual effect from PTA regression onto markers.

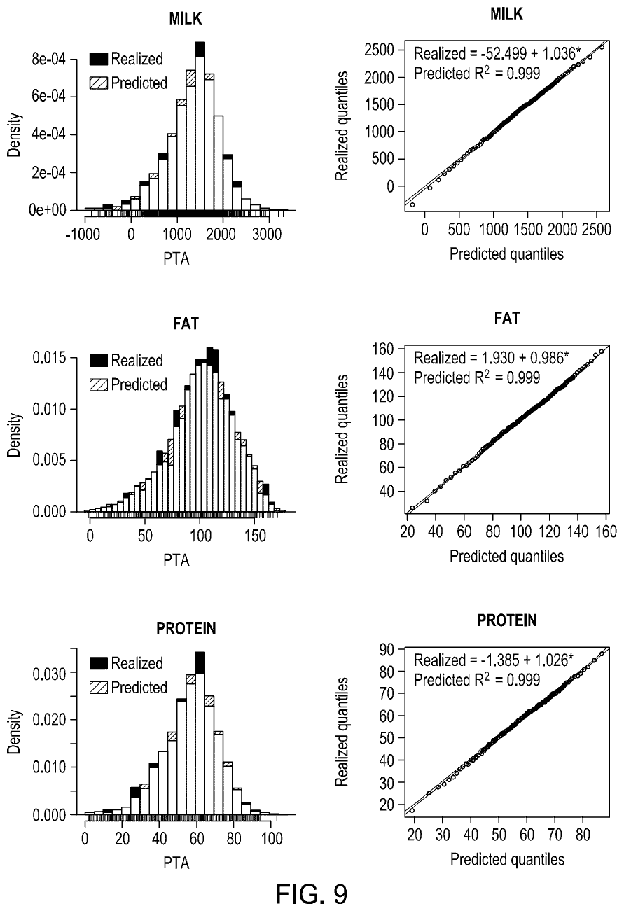

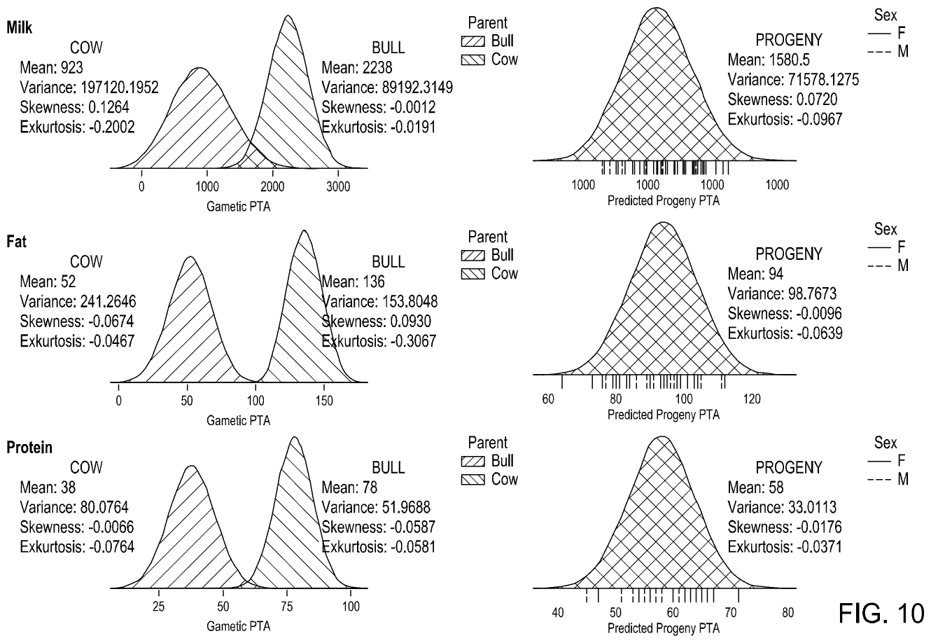

After dissecting genomic values in the haplotypic and gametic levels, the predictive ability of the invention was tested for progeny distributions. For progeny predictions, PTAs were used in replacement of DGVs assuming polygenic effects to contribute negligible variance to progeny PTAs and skewness and kurtosis of PTAs being interchangeable with those of DGVs given their property of being scale-free. Predicted and realized quantiles of progeny distributions were compared across 122 families comprised of 44 bulls, 103 cows and 1,563 offspring (see FIG. 9). An average correlation of 99.9% between predicted and realized quantiles was found, validating that the invention achieves high accuracy in the prediction of progeny distributions. The family with the largest number of offspring (n=42) was selected to further illustrate how well the realized progeny follows the predicted distribution (see FIG. 10). The PTAs of realized progeny followed closely the predicted PTA distribution.

FIG. 9 shows validation of predictions of progeny distributions for milk, fat and protein yield in 122 families. The rug plots under the histograms indicate observations of real progenies. The blue line in the quantile-quantile plots represents the 0/1 (45 degrees) regression line, whereas the red line represents the regression fitted to the data.

FIG. 10 shows Predicted x Realized progenies of a family including 42 offspring. Density plots on the left show the predicted PTA distributions of oocytes and sperm cells in the parents. The density plot on the right side shows the predicted distribution of PTAs in the progeny. Realized progenies are shown in the rug plot below the predicted density.

The visualization of haplotype effects has important implications to mating allocations. First, as also shown by Cole and Null (2013), elite animals are not exempt from carrying haplotypes with negative net DGVs, and thus pairing them with mates having haplotypes with positive net DGVs for the same chromosome pair is desirable. Second, since haplotypes with high positive net DGV can still present negative segments, finding mates that are further complementarily positive at those loci while also having positive net DGV may result in greater genetic gains. Third, heterozygosity at multiple major QTL with opposing effects within a chromosome pair could lead to recombinant haplotypes with highly positive or highly negative net DGV even if the source parental haplotypes have net DGV close to zero. This last observation predicts that exceptional recombinant haplotypes could still emerge from bulls and cows with average merit.

Visualizing the effect of major QTLs on the distributions of gametes is extremely helpful in detecting in which chromosome pairs lie the weaknesses and strengths in the transmitting ability of an animal. Also, by learning which chromosome pairs should be improved to maximize genetic gain, one can use chromosomal complementarity between animals in mating allocations. This is further facilitated by the approximation of progeny distributions from parental gametic moments. Here, the distribution of recombinant haplotypes and gametes revealed that assuming normally distributed DGVs or PTAs is not appropriate if the trait is influenced by major QTLs. For the three traits investigated here, QTLs at chromosomes 5, 6 and 14 had a significant role in distorting the shapes of distributions away from the normal distribution. Major genes mapping to these QTL regions include MGST1 (Littlejohn et al., 2016) on chromosome 5, CSN2 (Gallinat et al., 2013) and GC (Lee et al., 2021) on chromosome 6, and DGAT1 (Grisart et al., 2004) on chromosome 14, and their effects on multiple traits in Holstein cattle have been exhaustively studied (Jiang et al., 2019). Of note, the solution for allele substitution effects was based on a SNP-BLUP or ridge regression type model, which may be considered conservative since it exerts strong shrinkage on marker effects. The use of regression models that allow for marker-specific variances such as Bayes A (Meuwissen et al., 2001), or variable selection models like Bayes B (Meuwissen et al., 2001), C (Habier et al., 2011), R (Erbe et al., 2012) or Bayesian LASSO (Park & Casella, 2008), might accentuate further deviations of gametes from the normal distribution, as found in simulations (See Example 2).

Ultimately, prediction of progeny distributions using the methods of the invention should serve the purpose of improving mating optimizations and thus accelerate genetic progress. While parent averages have been classically used at the core of mating allocation scores in dairy cattle, Santos et al. (2019) proposed a selection index, namely relative PTA (RPTA), that includes variability information:

$${SCORE}_{i}^{( RPTA )} = {PTA}_{i} + z {\hat{σ}}_{i}$$

Where z is the selection intensity applied to the next generation expressed as the number of standard deviations between the population average and the average of selected individuals. A way to build on that index is to incorporate data from skewness and kurtosis as follows:

$${SCORE}_{i}^{( MPTA )} = {PTA}_{i} + z {\hat{σ}}_{i} [ 1 + {\hat{S}}_{i}^{3} - ( {\hat{K}}_{i}^{4} - 3 ) ]$$

Where MPTA stands for moments-based PTA. In the case of selection of individuals, this score helps ranking up genitors that have higher probability of generating sets of recombinant haplotypes with greater net DGV. For mating allocations, this score can be used to distinguish matings that have a higher probability of yielding higher progeny PTAs among those with similar parent averages, which would indirectly inform better haplotypic complementarity.

For the specific goal of mating for elite animals, allocations will likely shift focus to the generation of outlier progeny rather than maximizing genetic merit population-wise. In this context, the probability of offspring surpassing a desired threshold PTA may be a useful alternative allocation score:

$${SCORE}_{progeny}^{( P )} = Pr ( {PTA}_{progeny} > threshold \vert {moments}_{progeny} )$$

This score is easily approximated with the invention using the Edgeworth expansion with estimated progeny moments. Alternatively, for matings with small absolute values for skewness and excess kurtosis (say <0.1), a normal approximation could be used. Low probability matings can be avoided altogether, and the probability score itself informs how many embryos on average one should produce to pass the threshold.

To maximize usefulness of the invention, a pipeline that automatically processes newly genomically tested animals can be established. Once the DGVs of chromosomal segments are differentiated and gametic statistics are calculated, the results can be stored in a SQL server for later display in a web application, as well as for running linear optimizations of matings using allocation scores. Thus, apart from browsing individual animal data, selection decisions and mating plans are achievable within a single platform. Furthermore, a Hidden Markov Model (HMM) mapping identical-by-descent (IBD) segments between pairs of haplotypes can be implemented such that inheritance of haplotype segments of interest can be traced in a pedigree or used to reveal autozygosity through the identification of runs of homozygosity (ROH). This rich layering of data allows for a fine control of the genomic inventory and facilitates management of available genetic resources. Finally, the invention can be used to predict gametic and progeny distributions of ancestry proportions in crossbred populations.

### Example 2

### Simulated SNP Effects

To test the impact of the distribution of allele substitution effects (as a proxy for genetic architecture) on the variation of skewness and kurtosis of gametes, a range of simulations was performed, including the following distributions of SNP effects:

SNP-BLUP (normal distribution):

$$α ∼ N ( 0 , 1 / N ) .$$

BayesA (normal distribution with heterogeneity of variance):

αⱼ˜N(0,σⱼ²) and σⱼ²˜inv−χ²(ν), with ν=20 and of scaled as σⱼ²/Σσⱼ².

BayesB (normal distribution with heterogeneity of variance+point mass at zero):

αⱼ=0 with probability π and aⱼ˜N(0,σⱼ²) with probability 1−π. We assumed π=0.9 or π=0.99, and of was sampled as described for BayesA.

BayesC (normal distribution+point mass at zero)

Same as BayesB but with a common marker variance of 1/[(1−π)N].

BayesR (mixture of three normal distributions+point mass at zero)

αⱼ=0 with probability π₁, αⱼ˜N(0, 10⁻⁴) with probability π₂, αⱼ˜N(0, 10⁻³) with probability π₃, and aⱼ˜N(0, 10⁻²) with probability σ₄. For this model, two analyses were performed. In the first, the probabilities were set to σ₁=0.9000, σ₂=0.0920, σ₃=0.0074 and σ₄=0.0006, as found for milk traits in Holstein by Erbe et al. (2012). In the second, the probabilities were modified to 11=0.99000, 12=0.0087125, 13=0.0012500 and T4=0.0000375, following simulations performed by Breen et al. (2022).

LASSO (double exponential distribution)

|αⱼ|˜Exp(λ). The effect was multiplied by −1 with probability 0.5. The rate parameter was set to λ=5.81 (Bennewitz & Hayes, 2010; Zeng et al., 2013).

These distributions were selected because they mirror priors commonly used in regression models for genomic predictions. Each scenario was replicated 5 times, and marker effects were scaled by \sqrt{Σαⱼ²} to ensure that the total marker variance was 1 in each replica. These simulated effects were then used with the real Holstein genotypes and the gamete simulator described above. Impact of genetic architecture on gametic diversity

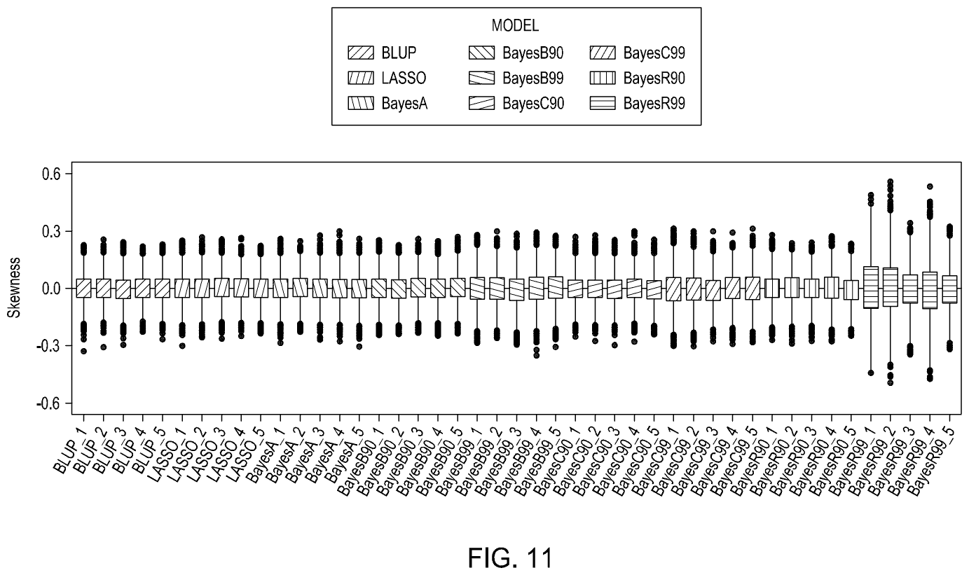

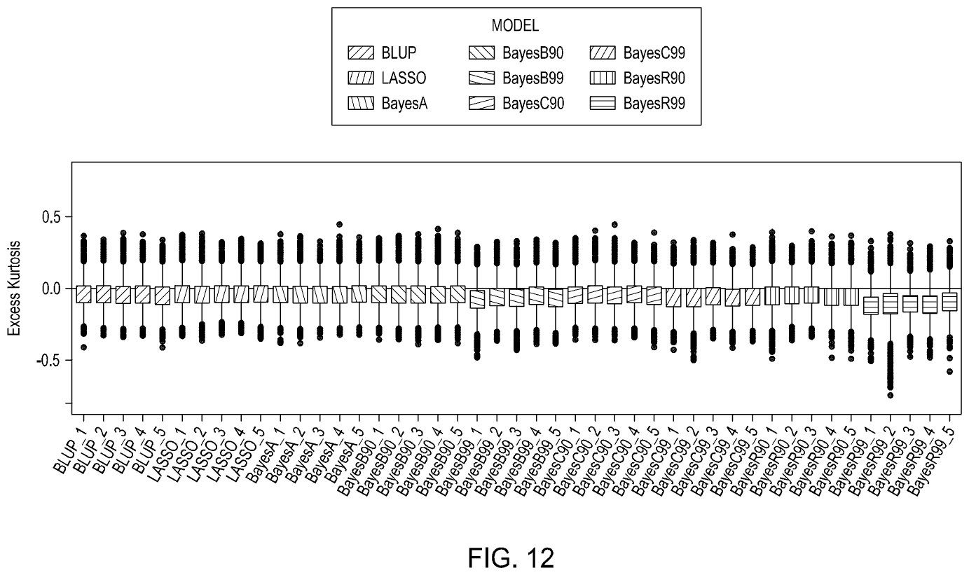

The effect of different distributions of marker effects are shown in FIG. 11 and FIG. 12. In short, the greater the contrast between major QTLs and genome-wide effects (or conversely the less shrinkage imposed to solutions of SNP effects) the greater was the variation in skewness and the lower was the kurtosis. This further confirms that deviations of gametic diversity from normal distributions are mainly caused by major QTLs.

FIG. 11 shows the distribution of gametic skewness for different simulated distributions of SNP effects.

FIG. 12 shows the distribution of gametic (excess) kurtosis for different simulated distributions of SNP effects.

### Example 3

### Improving Genetic Merit Using Chromosomal Segment Complementarity

Some alleles with large effects on phenotypes or underlying special Mendelian traits can have low frequency in a population. Typically, animals that are carriers of such alleles pertain to unselected or less selected genetic lines and consequently may not be commonly mated with top genetics animals. For example, only 1.1% of the US Holstein cattle carry the POLLED allele causing natural absence of horns, and out of the top 100 proven Holstein bulls in the latest international evaluation for Total Performance Index (TPI) published in December 2023 by the US Holstein Association only one was a carrier of the POLLED allele, ranking 67th place. Disbudding and dehorning are common practices in the management of dairy cattle that aim at reducing risks of animal and human injuries. However, those practices are not only costly but also represent a major animal welfare issue.

The instant invention can be used to aid in the creation of a lineup of elite genetics Holstein animals that carry the POLLED allele by promoting optimized matings that aim at haplotype complementarity. This could be achieved by first analyzing the DGVs of chromosomal segments of a lower TPI animal that is homozygous for the POLLED allele and screening through a database of high TPI animals to find those that have large positive DGV values for the chromosomal segments where the lower TPI animal has a negative DGV. Through the use of artificial reproductive technologies (ART) such as in vitro fertilization and cell culture, cell lines could be subjected to genotyping and chromosomal segment analysis to select for embryo transfer only those that carry the POLLED allele and that have much higher TPI values than the parent average. Once semen or oocytes can be collected from the born progeny, the process is then repeated one or two additional generations, until the first line up of elite POLLED animals is produced. Given the shortening of the cattle generation interval promoted by recent advancements in ART, each generation and selection cycle could be realized as fast as 17 months. The whole process would take then between three to four years, which represents a single natural mating generation interval in cattle.

Gene editing is an alternative technology that can modify a genome to introduce an allele like POLLED in a background genome with high TPI in a single generation. However, regulations and the public perception of the technology makes its use prohibitive in dairy cattle breeding. This makes the instant invention, coupled with ART, a competitive and public policy issue-free alternative to gene editing in terms of increasing the frequency of naturally occurring low frequency alleles in a population.

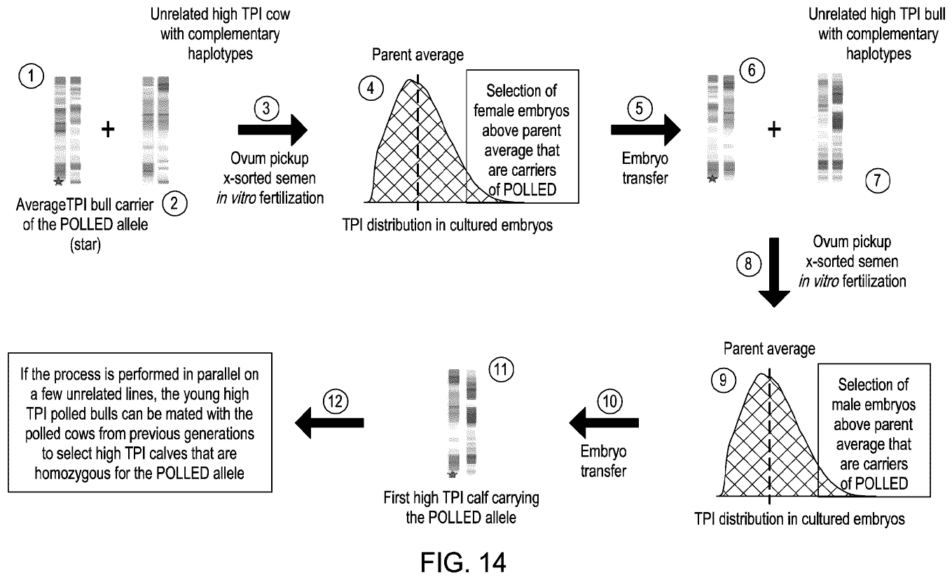

FIG. 14 is a depiction of the above-described process. Referring to FIG. 14, an average TPI bull carrier of the POLLED allele (1) (the star representing the chromosomal segment comprising the POLLED allele) and an unrelated high TPI cow (2) are selected as parents based on chromosomal segment complementarity (i.e., the chromosomal segments comprising negative DGVs in the average TPI bull carrier of the POLLED allele (1) (shown in shades of red) are complemented by positive DGVs in the corresponding chromosomal segments of the unrelated high TPI cow (2) (shown in shades of blue). The average TPI bull carrier of the POLLED allele (1) and the unrelated high TPI cow (2) are bred using ovum pickup and X chromosome-bearing sorted semen-sorted (3) to produce female cultured embryos. The embryos are then genotyped and the TPI distribution of the cultured embryos (4) is used to select embryos with TPI values greater than the parent average (represented by the dashed line) that are carriers of POLLED. Those selected embryos are then transferred into recipient females (5) to produce at least one heifer with improved recombinant chromosomal segments that is a carrier of POLLED (6). One can then select the heifer with improved recombinant chromosomal segments (6) as a parent to be mated with an unrelated high TPI bull with complementary chromosomal segments (7). The heifer with improved recombinant chromosomal segments (6) and the unrelated high TPI bull with complementary chromosomal segments (7) are bred using ovum pickup and Y chromosome-bearing sorted semen (8) to produce male cultured embryos. The male cultured embryos are then genotyped and the TPI distribution of the male cultured embryos (9) is used to select embryos with TPI values greater than the parent average (represented by the dashed line) that are carriers of POLLED. Those selected embryos are then transferred into recipient females (10) to produce at least one high TPI male with improved recombinant chromosomal segments that is a carrier of POLLED (11). The high TPI male with improved recombinant chromosomal segments that is a carrier of POLLED (11) can then be bred with POLLED cows from previous generations (12) to produce high TPI calves that are homozygous for the POLLED allele.

What we claim is:

1. A method of producing animal or plant progeny comprising
generating a phased genotype from a genotype of an animal or a plant in a population, wherein the phased genotype is comprised of a plurality of phased alleles;
multiplying each phased allele in the plurality of phased alleles by a marker effect to produce a direct genomic value (DGV) for each phased allele;
segmenting the phased genotype, wherein each of the segments is comprised of one or more phased alleles from the plurality of phased alleles;
calculating a DGV for each of the segments to provide either a positive or negative DGV for each of the segments;
selecting the animal or the plant as a parent based on the calculated DGV of one or more of the segments; and
producing progeny from the animal or the plant.

2. The method of claim 1, wherein the number of segments and the size of each of the segments are determined heuristically.

3. The method of claim 1, wherein the number of segments and the size of each of the segments are a function of the effective number of independent chromosomal segments.

4. The method of claim 1, wherein the DGV for each of the segments is calculated using a sliding window method.

5. The method of claim 4, wherein the sliding window method comprises selecting a step size.

6. The method of claim 1, further comprising a step of displaying the segmented, phased genotype, wherein segments comprising a positive DGV are visually differentiated from segments comprising a negative DGV.

7. The method of claim 6, wherein the segments comprising a positive DGV are visually differentiated from the segments comprising a negative DGV using a color gradient produced from a set of colors, wherein each color in the set represents a percentile of the distribution of DGVs for a segment in the population.

8. A method of producing animal or plant progeny comprising
estimating skewness or kurtosis of a distribution of DGVs of an animal's gametes or a plant's gametes using i) phased genotypes or linkage phase information and ii) marker recombination rates;
selecting the animal or the plant as a parent in a population based on the estimated skewness or kurtosis of the distribution; and
producing progeny from the animal or the plant.

9. The method of claim 8, wherein the estimated skewness is positive.

10. The method of claim 8, wherein the estimated kurtosis is less than 3.

11. The method of claim 8, wherein the estimated kurtosis is greater than 3.

12. The method of claim 8, wherein the step of selecting the animal or the plant is based on the estimated kurtosis comprises calculating excess kurtosis.

13. The method of claim 12, wherein the calculated excess kurtosis is negative.

14. The method of claim 12, wherein the calculated excess kurtosis is positive.

15. A method of producing progeny from a male and a female of an animal or plant species comprising
estimating skewness or kurtosis of a distribution of DGVs for the male's gametes and for the female's gametes using i) phased genotypes or linkage phase information and ii) marker recombination rates;
estimating a distribution of progeny DGVs, EBVs or PTAs using the estimated skewness or kurtosis for the male's gametes and the estimated skewness or kurtosis for the female's gametes;
selecting the male and the female as a mating pair based on the estimated distribution of progeny DGVs, EBVs or PTAs; and
producing progeny from the mating pair.

16. The method of claim 15, wherein the step of selecting the male and the female as a mating pair based on the estimated distribution of progeny DGVs, EBVs or PTAs comprises calculating the probability of producing progeny having a DGV, EBV or PTA exceeding a threshold DGV, EBV or PTA.

17. A method of producing progeny from a male and a female of an animal or plant species comprising
generating a phased genotype from a genotype of the male and a phased genotype from a genotype of the female, wherein each of the phased genotypes is comprised of a plurality of phased alleles;
for each of the phased genotypes, multiplying each phased allele in the plurality of phased alleles by a marker effect to produce a direct genomic value (DGV) for each phased allele;
segmenting each of the phased genotypes, wherein each segment is comprised of one or more phased alleles from the plurality of phased alleles;
for each of the phased genotypes, calculating a DGV for each of the segments to provide either a positive or a negative DGV for each of the segments;
selecting the male and the female as a mating pair based on the calculated DGV of one or more segments from the male and one or more segments from the female; and
producing progeny from the mating pair.

18. The method of claim 17, wherein in the step of selecting the male and the female as a mating pair, the one or more segments from the male and the one or more segments from the female are located on the same corresponding chromosome pair in the male and the female and i) the calculated DGV of the one or more segments from the male is a positive DGV, and the calculated DGV of the one or more segments from the female is a negative DGV or ii) the calculated DGV of the one or more segments from the male is a negative DGV, and the calculated DGV of the one or more segments from the female is a positive DGV.
